(19) United States
(12) Patent Application Publication
(10) Pub. No.: US 2025/0277017 A1
(43) Pub. Date: Sep. 4, 2025

(54) COMPOSITIONS AND METHODS FOR TREATING EYE DISEASES

(71) Applicant: BOARD OF REGENTS, THE UNIVERSITY OF TEXAS SYSTEM, Austin, TX (US)

(72) Inventors: Jean X. JIANG, San Antonio, TX (US); Sumin GU, San Antonio, TX (US); Manuel RIQUELME, San Antonio, TX (US)

(21) Appl. No.: 18/858,834

(22) PCT Filed: Apr. 28, 2023
(86) PCT No.: PCT/US2023/066402

§ 371 Date: Oct. 22, 2024

Related U.S. Application Data
(60) Provisional application No. 63/336,511, filed on Apr. 29, 2022.

(51) Int. Cl. C07K 16/18; A61K 39/00; A61P 27/02; A61P 27/06

(52) U.S. Cl. CPC C07K 16/18; A61P 27/02; A61P 27/06; A61K 2039/505; A61K 2039/54; C07K 2317/24; C07K 2317/565

(57) ABSTRACT

Disclosed herein are antibodies that bind to connexin 43 hemichannels to inhibit or block channel opening. Also disclosed herein are methods for treating or preventing eye diseases, eye injuries, glaucoma, macular degeneration, diabetic retinopathy, retina injury, and optical nerve damage and degeneration with antibodies that inhibit or block Cx43 hemichannel opening.

### CROSS REFERENCE TO RELATED APPLICATIONS

This application claims the benefit of the filing date of U.S. Provisional Application No. 63/336,511, filed on Apr. 29, 2022. The content of these earlier filed applications is hereby incorporated by reference in their entirety.

### REFERENCE TO A SEQUENCE LISTING

The present application contains a Sequence Listing that is submitted concurrent with the filing of this application, containing the file name “21105_0090P1_SL.xml,” created on Apr. 25, 2023, and having a size of 77,824 bytes, and is hereby incorporated by reference pursuant to 37 C.F.R. § 1.52(e)(5).

### BACKGROUND

Glaucoma is eye disease that damages the optical nerve and is a leading cause of sight impairment and blindness. About 2.3 million people have glaucoma and 10% of patients suffer from vision impairment and 120,000 are blind. Currently, there is no cure for glaucoma, and stabilizing intraocular pressure to a lower level as an early treatment is the only therapeutic means available to reduce the damage to the optical nerve and protect vision. A need also exists for treating glaucoma.

### SUMMARY OF THE INVENTION

Disclosed herein are methods of treating or preventing glaucoma in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing macular degeneration in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing an eye disease or an eye injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing diabetic retinopathy in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing retinal injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing optical nerve damage or degeneration in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing glaucoma in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing macular degeneration in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing an eye disease or an eye injury in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing diabetic retinopathy in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing retinal injury in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing optical nerve damage or degeneration in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing glaucoma in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are method of treating or preventing macular degeneration in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of treating or preventing an eye disease or an eye injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of treating or preventing diabetic retinopathy in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of treating or preventing retinal injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of treating or preventing optical nerve damage or degeneration in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of reducing retinal ganglion cell loss or reducing retinal ganglion apoptosis, the methods comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of protecting or preserving retinal ganglion cell axon, somata or dendrites in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing reactive gliosis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing microglia activation or neuroinflammation in the retina in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing optical nerve damage and degeneration in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of protecting visual function in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing retinal ganglion cell loss or reducing retinal ganglion apoptosis, the methods comprising administering to a subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of protecting or preserving retinal ganglion cell axon, somata or dendrites in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing reactive gliosis in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing microglia activation or neuroinflammation in the retina in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing optical nerve damage and degeneration in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of protecting visual function in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing retinal ganglion cell loss or reducing retinal ganglion apoptosis, the methods comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of protecting or preserving retinal ganglion cell axon, somata or dendrites in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of reducing reactive gliosis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of reducing microglia activation or neuroinflammation in the retina in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of reducing optical nerve damage and degeneration in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of protecting visual function in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

Disclosed herein are methods of reducing or ameliorating one or more symptoms of an eye disease or an eye injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing or ameliorating one or more symptoms of an eye disease or an eye injury in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing or ameliorating one or more symptoms of an eye disease or an eye injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 and a variable light chain comprising a sequence having at least 90% identity to a sequence set forth in SEQ ID NO: 60.

In further embodiments, the anti-connexin Cx43 hemichannel blocking antibody or fragments thereof for use according to the embodiments can be any of those described in international (PCT) patent publication no. WO 2015-027120 or WO 2017-147561, which are incorporated herein by reference for their teaching of anti-connexin Cx43 hemichannel blocking antibodies, fragment thereof, vectors and cells for making or expressing antibodies.

Other aspects of the invention are discussed throughout this application. Any aspect discussed with respect to one aspect of the invention applies to other aspects of the invention as well and vice versa. Each aspect described herein is understood to be aspects of the invention that are applicable to other aspects of the invention. It is contemplated that any aspect discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-D show that the haplosufficiency of Cx43 reduces retinal function loss and retinal injury after optical never crush (ONC). FIG. 1A shows PhNR of photopic ERG from Cx43 (+/−) and WT mice before and 4 weeks after ONC. FIG. 1B shows a histogram of the PhNR amplitude (from the baseline to the trough of the negative response following the positive b-wave) under different conditions. FIG. 1C shows the thickness of retina examined by OCT from Cx43 (+/−) and WT mice before and 4 weeks after ONC. FIG. 1D shows a histogram of the thickness of retinal under different conditions.

FIGS. 2A-D show that inhibition of Cx43 hemichannels by M1 antibody protects visual function after ONC. FIG. 2A shows a histogram of the pSTR peak amplitude under different conditions. FIG. 2B shows a histogram of the PhNR amplitude (from the baseline to the trough of the negative response following the positive b-wave) under different conditions. FIG. 2C shows the thickness of retina tested by OCT from WT before and 4 weeks after ONC with or without M1 antibody. FIG. 2D shows a histogram of the thickness of retinal under different conditions.

FIGS. 3A-E shows that the M1 antibody reduces retinal ganglion cell loss. FIG. 3A) shows mouse retinal sections immunofluorescence stained with BRN3A antibody and counterstained with DAPI 4 weeks after ONC with or without treatment with the M1 antibody. FIG. 3B shows the number of BRN3A-positive RGCs in the ganglion cell layer (GCL) of retinas. FIG. 3C shows the number of BRN3A-positive RGCs in the GCL of retinas from Cx43 (+/−) and WT mice. FIG. 3D shows images of whole-mount mouse retinas with BRN3A-labeled RGCs 4 weeks after ONC with or without treatment with the M1 antibody. The images and measurements were taken from the midperiphery of the retina, 1.5-2.0 mm from the optic disk (inset). FIG. 3E shows that RGCs were quantified from 4 quadrants of each imaging field area and averaged for both control and experimental conditions.

FIG. 4 shows that M1 antibody reduces apoptosis after ONC. Fluorescence images of retinal sections labeled for Tunel (red) and DAPI (blue) 4 weeks after ONC with or without treatment with the M1 antibody.

FIGS. 5A-B show that the M1 antibody preserves RGC axon, somata and dendrites after ONC. FIG. 5A shows fluorescence images of whole-mount mouse retinas with SMI-32-labeled axon, RGC somata and dendrites 4 weeks after ONC with or without treatment with the M1 antibody. FIG. 5B shows fluorescence images of whole-mount Cx43 (+/−) and WT mouse retinas with SMI-32 labeled axon, RGC somata and dendrites 4 weeks after ONC. The bar graphs (lower panels) below each image in FIG. 5A and FIG. 5B show the numbers of axon bundles in a quarter of neurons (lower panels). *P<0.05. n=3-4.

FIGS. 6A-G show that the M1 antibody reduces reactive gliosis after ONC. FIG. 6A shows fluorescence images of retinal sections labeled for Cx43 (green), GFAP (red) and DAPI (blue) 4 weeks after ONC with or without treatment with the M1 antibody. FIG. 6B shows the expression of GFAP in the mouse retina under the same condition described in FIG. 6A. FIG. 6C shows Cx43 expression in the retinas from C57BL/6 mice under the same conditions described in FIG. 6A. Protein extracts of mouse retinas 4 weeks after ONC with or without treatment with the M1 antibody were immunoblotted with anti-Cx43CT (FIG. 6D) or GFAP (FIG. 6F) antibody and GAPDH antibody and quantified (FIG. 6E and FIG. 6G), respectively.

FIG. 7 shows that M1 antibody reduces microglia and neuroinflammation after ONC. Fluorescence images of mouse retinal sections labeled for iNOS (green), CD68 (red) and DAPI (blue) 4 weeks after ONC with or without treatment with the M1 antibody.

FIG. 8 shows that Cx43 hemichannel opening in astrocytes induced by inflammatory factor, interleukin-1 beta/TNG-alpha is inhibited by an anti-Cx43 hemichannel blocking antibody (E2). Primary astrocyte cells were plated at low density (8000 cells/cm²) and cultured with DMEM/F12 medium plus 10% FBS until confluence. A mixture of interleukin-1β (IL-1β, 10 ng/ml) and tumor necrosis factor-α (TNF-α; 10 ng/ml) IL-1β was added to medium, and cells were incubated for 24 hours. Before adding mixture, anti-Cx43 hemichannel blocking antibody (E2) was added to medium, and cells were incubated for 30 minutes prior to addition of EtBr (50 μM) for 5 minutes. The data were presented as Mean±SEM. Two way ANOVA. **P<0.01.

FIG. 9 shows stained opening of astrocyte Cx43 in response to cytokines IL-1beta/TNF-alpha treatment. Even after removal of IL-1beta/TNF-alpha, hemichannel-mediated dye uptake was shown 4 hour post treatment. After 24 hours of post-treatment, dye uptake level was close to basal level. Fluorescence images were taken by using Countess™ cell counting chamber slides using a fluorescence microscope (Keyence BZ-X710), and the percentage of positive staining cells was quantified. The data are presented as Mean±SEM. Two way ANOVA, *P<0.05.

FIG. 10 shows inhibition of astrocyte Cx43 hemichannel opening protects retinal ganglion cells (RGCs). Primary astrocyte cells were plated at low density (2×10⁵ cells/cm²) and incubated with DMEM/F12 medium plus 10% FBS until confluence. A mixture of IL-1B (10 ng/mL) and TNF-α (10 ng/ml) was added to medium, and cells were incubated for 24 hours. Before adding mixture, anti-Cx43 hemichannel blocking antibody (E2) was added to primary astrocytes and incubated for 30 minutes. Then medium was replaced by previously 48-hours cultured medium and the conditioned medium (CM) was collected after 24 hours. The RGC-like R28 cells were cultured with CM for 48 hours. The R28 cells were collected and centrifuged. The cell pellets were resuspended in Annexin V Binding Buffer and further processed using FITC-Annexin V Apoptosis Detection Kit (BioLegend, San Diego, CA, USA) according to the manufacturer instructions. Fluorescence images were taken by using Countess™ cell counting chamber slides using a fluorescence microscope (Keyence BZ-X710), and the percentage of positive staining cells was quantified. Compared to the control group, R28 cells were incubated with CM collected from astrocytes treated with IL-1B/TNF-alpha intervention for 48 hours, the percentage of apoptotic RGC cells increased and this increase was ablated by an anti-Cx43 blocking antibody (E2). Early apoptosis is indicated by Annexin V-positive cells and late is indicated by PI-positive stained cells. The data are presented as Mean±SEM. Two way ANOVA, **P<0.01; ****P<0.0001.

FIG. 11 shows inhibition of Cx43 hemichannel opening in astrocytes protect RGC dendritic damages caused by inflammatory cytokines IL-1beta/TNF-alpha. It was tested whether inflammatory cytokines, IL-1beta/TNF-alpha, that open astrocyte Cx43 hemichannels and release ATP and glutamate causes RGC dendritic damages using primary astrocyte cells that were plated at low density (2×10⁵ cells/cm²) and incubated with DMEM/F12 medium plus 10% FBS until confluence. A mixture of IL-1B (10 ng/mL) and TNF-α (10 ng/mL) was added to medium, and cells were incubated for 24 hours. Before adding mixture, an anti-Cx43 hemichannel blocking antibody (E2) was added to primary astrocytes and incubated for 30 minutes. The medium was replaced by previously 48-hours cultured medium and the conditioned medium (CM) was collected after 24 hours. The RGC-like R28 cells were cultured with CM for 48 hours. The R28 cells were fixed with 4% PFA and stained with anti-F-actin (1:200 dilution). Fluorescence images were taken by using Countess™ cell counting chamber slides using a fluorescence microscope (Keyence BZ-X710), and the percentage of dendrites were quantified. The data are presented as Mean±SEM. Two way ANOVA. **P<0.01; ****P<0.0001.

FIG. 12 shows that Cx43 hemichannel releases ATP and extracellular ATP increases RGC-like R28 cell apoptosis. Primary astrocyte cells were plated at low density (2×10⁵ cells/cm²) and incubated with DMEM/F12 medium plus 10% FBS until confluence. A mixture of IL-1β (10 ng/mL) and TNF-α (10 ng/ml) was added to medium, and cells were incubated for 24 hours. Before adding mixture, an anti-Cx43 hemichannel blocking antibody (E2) was added to primary astrocytes and incubated for 30 minutes. Then medium was replaced by previously 48-hours cultured medium and the conditioned medium (CM) was collected after 24 hours. Some CM were treated with apyrase (5 units/ml), an ATP hydrolyase to degrade ATP. The R28 cells were cultured with CM for 48 hours and centrifuged. The cell pellets were resuspended in Annexin V Binding Buffer and further processed using FITC-Annexin V Apoptosis Detection Kit (BioLegend, San Diego, CA, USA) according to the manufacturer instructions. Fluorescence images were taken by using Countess™ cell counting chamber slides using a fluorescence microscope (Keyence BZ-X710), and the percentage of positive staining cells was quantified. Early apoptosis is indicated by Annexin V-positive cells and late is indicated by PI-positive stained cells. The data were presented as Mean±SEM. Two way ANOVA, *P<0.05.

FIG. 13 shows inhibition of Cx43 hemichannel opening by a monoclonal anti-Cx43 hemichannel blocking antibody (the M1 antibody) protects RGC degradation induced by increased intraocular pressure (IOP) in silicon oil (SO) injection mouse model. After three weeks of SO injection, optical coherence tomography (OCT) imaging showed that the thickness of GCC (comprising retinal nerve fiber layer (RNFL), ganglion cell layer (GCL) and inner plexiform layer (IPL)) was significantly decreased and that this decrease was partly, but significantly reversed with a single intravitreal injection of M1 antibody (25 mg/kg) two days after the SO. The data were presented as Mean±SEM. Two way ANOVA, **P<0.01. ****P<0.001.

FIG. 14 shows that inhibition of Cx43 hemichannels by an anti-Cx43 hemichannel antibody (the M1 antibody) protects RGC degeneration induced by elevated IOP in silicon oil (SO) injection mouse model. After three weeks of SO injection, whole-mount mouse retinas were labeled with BRN3A antibody (1:100 dilution). The images were captured around the mid-periphery regions of the retina (1.5-2 mm from the optic disk). The loss of RGC cells was shown after 3 weeks of SO injection and this loss was partly, but significantly reversed with a single intravitreal injection of the M1 antibody (25 mg/kg) two days after the SO. The data were presented as Mean±SEM. Two way ANOVA. *P<0.05. ***P<0.01.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list. The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

“Treatment” and “treating” refer to administration or application of a therapeutic agent (e.g., an anti-Cx43 antibody described herein) to a subject or performance of a procedure or modality on a subject for the purpose of obtaining a therapeutic benefit of a disease or health-related condition. For example, a treatment may include administration of a pharmaceutically effective amount of an antibody or fragment thereof that inhibits or blocks the opening of the Cx43 hemichannel.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be an eye disease or an eye injury. In some aspects, the disease, disorder, and/or condition can be glaucoma, macular degeneration, diabetic retinopathy, retina injury, and optical nerve damage and degeneration.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a condition, disease or disorder. The term “patient” includes human and veterinary subjects. In some aspects of the disclosed methods, the “patient” has been diagnosed with an eye disease, an eye injury, glaucoma, macular degeneration, diabetic retinopathy, retina injury, or optical nerve damage or degeneration. In some aspects of the disclosed methods, the “patient” has been diagnosed with a need for treatment (e.g., treatment for an eye disease, an eye injury, glaucoma, macular degeneration, diabetic retinopathy, retina injury, or optical nerve damage or degeneration), such as, for example, prior to the administering step.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the(S) configuration. In some aspects, the amino acids are in the(S) or L-configuration. If non-naturally occurring side chains are used, non-amino acid substituents may be used, for example to prevent or retard in vivo degradation.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

As used herein, the term “MIH” refers to an antibody that was cloned from hybridoma clones. “M1” refers to hybridoma monoclonal 1, and “H” refers to the variable heavy chain. As used herein, the term “MIM7K” refers to the variable light chain identified from hybridoma clones M1 and M7.

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256:495-497, 1975), or by recombinant DNA methods.

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as antibodies, antibody fragments, polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “providing” is used according to its ordinary meaning “to supply or furnish for use.” In some aspects, the protein is provided directly by administering the protein, while in other aspects, the protein is effectively provided by administering a nucleic acid that encodes the protein. In certain aspects, the invention contemplates compositions comprising various combinations of nucleic acid, antigens, peptides, and/or epitopes.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Glaucoma is chronic optic neuropathy with characteristic patterns of optic nerve head excavation and retinal ganglion cell (RGC) loss. One strategy to effectively treat glaucoma is to protect RGCs and thereby preserve vision. Preventing the death of RGCs or regenerating the RGCs can protect RGCs. Astrocytes are the most prevalent glial cell type in the optic nerve head and retina. Connexin 43 (Cx43) is the most abundant gap junction protein in the CNS and is primarily expressed on astrocytes. The opening of Cx43 hemichannels during neuronal injury promotes gliosis and secondary injury. Therefore, inhibition of Cx43 hemichannel opening by the anti-Cx43 antibody (e.g., the M1 antibody) is a viable strategy in treating glaucoma and alleviating optical nerve degeneration. In addition to glaucoma, abnormal opening of Cx43 channels has also been reported to be involved in other eye diseases including but not limited to macular degeneration, diabetic retinopathy, retinal injury and degeneration. Thus, inhibition of Cx43 hemichannel opening by the anti-Cx43 antibody (e.g., the M1 antibody) can also be useful in treating macular degeneration, diabetic retinopathy, retinal injury and degeneration.

### Antibodies

Disclosed herein are anti-connexin 43 antibodies (also referred to herein as “anti-Cx43 antibodies” or “anti-CX43 antibodies”) that can be used in one or more of the methods disclosed herein. Disclosed herein are anti-connexin 43 antibodies that can inhibit or block the opening hemichannels, and in particular, Cx43 hemichannels. As used herein, an anti-Cx43 hemichannel blocking antibody can be referred to as “M1 antibody”. An example of identifying and isolating a monoclonal antibody is described below.

The term “CDR” as used herein refers to a Complementarity Determining Region of an antibody variable domain. Systematic identification of residues included in the CDRs have been developed by Kabat et al. (1991, Sequences of Proteins of Immunological Interest, 5th Ed., United States Public Health Service, National Institutes of Health, Bethesda). Variable light chain (VL) CDRs are herein defined to include residues at positions 27-32 (CDR1), 50-56 (CDR2), and 91-97 (CDR3). Variable heavy chain (VH) CDRs are herein defined to include residues at positions 27-33 (CDR1), 52-56 (CDR2), and 95-102 (CDR3). In some aspects, variable light chain (VL) CDRs are herein defined to include residues at positions 27-32 (CDR1), 50-56 (CDR2), and 91-97 (CDR3) of SEQ ID NO: 60. In some aspects, variable heavy chain (VH) CDRs are herein defined to include residues at positions 27-33 (CDR1), 52-56 (CDR2), and 95-102 (CDR3) of SEQ ID NO: 58.

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent (e.g., SEQ ID NOs: 19, 20, 21, 31, 32 or 33) or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR (e.g., SEQ ID NOs: 19, 20, 21, 31, 32 or 33). For example, a “variant CDR” can be a sequence that contains 1, 2, 3 or 4 amino acid changes as compared to the parent or reference CDR of the invention, and shares or improves biological function, specificity and/or activity of the parent CDR.

Disclosed herein are anti-connexin 43 (Cx43) hemichannel blocking antibodies comprising a first VH CDR corresponding to SEQ ID NO: 19 or a fragment thereof, a second VH CDR corresponding to SEQ ID NO: 20 or a fragment thereof, a third VH CDR corresponding to SEQ ID NO: 21 or a fragment thereof, a first VL CDR corresponding to SEQ ID NO: 31 or a fragment thereof, a second VL CDR corresponding to SEQ ID NO: 32 or a fragment thereof, and a third VL CDR corresponding to SEQ ID NO: 33 or a fragment thereof. In some aspects, the anti-Cx43 hemichannel blocking antibody or fragment thereof can be a humanized antibody. In some aspects, the anti-Cx43 hemichannel blocking antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 31, a second VL CDR corresponding to SEQ ID NO: 32, and a third VL CDR corresponding to SEQ ID NO: 33. In some aspects, the anti-Cx43 hemichannel blocking antibody can be an IgG class of antibody, wherein the IgG class antibody can be an IgG1, IgG2, IgG3, or IgG4 class antibody. In some aspects, the anti-Cx43 hemichannel blocking antibody comprises a VH amino acid sequence at least 90% identical to SEQ ID NO: 58 or a fragment thereof and/or a VL amino acid sequence at least 90% identical to SEQ ID NO: 60 or a fragment thereof. In some aspects, the anti-Cx43 hemichannel blocking antibody may comprise a VH amino acid sequence according to SEQ ID NO: 58 or a fragment thereof and/or a VL amino acid sequence according to SEQ ID NO: 60 or a fragment thereof.

Disclosed herein are recombinant anti-connexin 43 (Cx43) hemichannel blocking antibodies or fragments. In some aspects, the anti-Cx43 hemichannel blocking antibody comprises a first VH CDR corresponding to SEQ ID NO: 19 or a fragment thereof, a second VH CDR corresponding to SEQ ID NO: 20 or a fragment thereof, a third VH CDR corresponding to SEQ ID NO: 21 or a fragment thereof, a first VL CDR corresponding to SEQ ID NO: 31 or a fragment thereof, a second VL CDR corresponding to SEQ ID NO: 32 or a fragment thereof, and a third VL CDR corresponding to SEQ ID NO: 33 or a fragment thereof. In some aspects, the anti-Cx43 hemichannel blocking antibody or fragment thereof can be a humanized antibody. In some aspects, the anti-Cx43 hemichannel blocking antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 31, a second VL CDR corresponding to SEQ ID NO: 32, and a third VL CDR corresponding to SEQ ID NO: 33. In some aspects, the anti-Cx43 hemichannel blocking antibody can be an IgG class of antibody, wherein the IgG class antibody can be an IgG1, IgG2, IgG3, or IgG4 class antibody. In some aspects, the anti-Cx43 hemichannel blocking antibody comprises a VH amino acid sequence at least 90% identical to SEQ ID NO: 58 or a fragment thereof and/or a VL amino acid sequence at least 90% identical to SEQ ID NO: 60 or a fragment thereof. In some aspects, the anti-Cx43 hemichannel blocking antibody may comprise a VH amino acid sequence according to SEQ ID NO: 58 or a fragment thereof and/or a VL amino acid sequence according to SEQ ID NO: 60 or a fragment thereof.

Disclosed herein are anti-Cx43 hemichannel blocking antibodies directed against hemichannel polypeptides, and nucleic acid molecules encoding said anti-Cx43 hemichannel blocking antibodies. In some aspects, the anti-Cx43 hemichannel blocking antibody can bind an epitope having an amino acid sequence of FLSRPTEKTI (SEQ ID NO: 13), KRDPCPHQVD (SEQ ID NO: 14), or LSAVYTCKR (SEQ ID NO: 15). In some aspects, the anti-Cx43 hemichannel blocking antibody can bind an epitope having an amino acid sequence of FLSRPTEKTI (SEQ ID NO: 13).

In some aspects, a first heavy chain region can comprise an amino acid sequence having an amino acid sequence of residues 13 to 37 of SEQ ID NO: 2; a second heavy chain region having an amino acid sequence corresponding to residues 46 to 66 of SEQ ID NO: 2; and a third heavy chain region comprising an amino acid sequence having an amino acid sequence of residues 97 to 116 of SEQ ID NO: 2. SEQ ID NO: 2:

EVQLEQPGAELVKPGASVKLSCKASGYTFTSYYMYWVKQRPGQG
LEWIGGINPSNGGTNFNEKFKNKATLTVDKSSSTAYMQLSSLTS
EDSAVYYCTREGNPYYTMNYWGQGTSVTVSSAKTTPPSVY.

In some aspects, the anti-Cx43 hemichannel blocking antibodies disclosed herein can include full-length antibodies, antibody fragments, single chain antibodies, bispecific antibodies, minibodies, domain antibodies, synthetic antibodies and antibody fusions, and fragments thereof.

In some aspects, the anti-Cx43 hemichannel blocking antibodies disclosed herein can include full length antibodies, antibody fragments, single chain antibodies, bispecific antibodies, minibodies, domain antibodies, synthetic antibodies and antibody fusions, and fragments thereof.

As used herein, the term “antigen” is a molecule capable of being bound by an antibody or T-cell receptor. In some aspects, binding moieties other than antibodies can be engineered to specifically bind to an antigen, e.g., aptamers, avimers, and the like.

The term “antibody” or “immunoglobulin” is used to include intact antibodies and binding fragments/segments thereof. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Typically, fragments compete with the intact antibody from which they were derived for specific binding to an antigen. Fragments include separate heavy chains, light chains, Fab, Fab′ F(ab′)2, Fabc, and Fv. Fragments/segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 79:315-21, 1990; Kostelny et al., J. Immunol. 148:1547-53, 1992.

The term “antibody” can include five different classes of human immunoglobulins, namely IgG, IgA, IgM, IgD, and IgE. In some aspects, the disclosed antibodies can be an IgG class of antibody which can be classified into the 4 subclasses of IgG1, IgG2, IgG3, and IgG4. In some aspects, the disclosed antibodies can be an IgA class of antibody which, can be classified into the 2 subclasses of IgA1 and IgA2. The basic structure of immunoglobulin is made up of 2 homologous L chains (light chains) and 2 homologous H chains (heavy chains). The immunoglobulin class and subclass are determined by H chains. In some aspects, the antibody or antibodies or variants or fragments thereof can be an IgG4.

While antigen-binding specificity is maintained, antibody stability of IgG4 can be improved. In some aspects, the antibody can be improved, for example, by substituting arginine (R) of IgG4 with glutamic acid (E), phenylalanine (F), isoleucine (I), asparagine (N), glutamine (Q), serine(S), valine (V), tryptophan (W), tyrosine (Y), lysine (K), threonine (T), methionine (M), or leucine (L).

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 19, 20, 21, 31, 32, and/or 33. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 19, 20, 21, 31, 32, and/or 33. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic-aspartate, glutamate; (2) basic-lysine, arginine, histidine; (3) nonpolar=alanine, valine, leucine, isoleucine, proline, phenylalanine, methionine, tryptophan; and (4) uncharged polar glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

In some aspects, the at least one amino acid change can be to substitute an NG motif (amino acid asparagine followed by a glycine). In some aspects, the glycine residue can be substituted or replaced with hydrophobic amino acid residue. In some aspects, the glycine residue can be substituted or replaced with alanine, aspartic acid, glutamic acid, or valine. In some aspects, the glycine residue can be substituted or replaced with arginine, lysine, or glutamine.

Many methods have been developed for chemical labeling and enhancement of the properties of antibodies and their common fragments, including the Fab and F(ab′)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteine residues on a peptide or in one of the disclosed antibodies or fragments thereof may be susceptible to forming disulfide linkages unless a reducing agent such as dithiothreitol (DTT) is added to the buffer or the cysteine residues can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

A “fragment antigen-binding fragment (Fab)” is a region of an antibody that binds to antigen. By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

A “CDR” or complementarity determining region is a region of hypervariability interspersed within regions that are more conserved, termed “framework regions” (FR). By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “antigen-binding portion” of an antibody (or simply “antibody portion”), as used herein, refers to one or more fragments of an antibody that retain the ability to specifically bind to an antigen (e.g., hemichannel). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL/VK, VH, CL and CH1 domains; (ii) a F(ab′)2 fragment, a bivalent fragment comprising two Fab fragments linked by a disulfide bridge at the hinge region; (iii) a Fab′ fragment, which can be an Fab with part of the hinge region (see, Fundamental Immunology (Paul ed., 3rd ed. 1993); (iv) a Fd fragment consisting of the VH and CH1 domains; (v) a Fv fragment consisting of the VL and VH domains of a single arm of an antibody; (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains.

The term “specifically binds” (or “immunospecifically binds”) is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5 fold, such as 10 fold, such as 25 fold, especially 50 fold, and particularly 100 fold or more, greater for a target molecule than its affinity for a non-target molecule. In some aspects, specific binding between an antibody or other binding agent and an antigen means a binding affinity of at least 106 M 1. Antibodies may, for example, bind with affinities of at least about 107 M 1, such as between about 108 M-1 to about 109 M-1, about 109 M-1 to about 1010 M-1, or about 10-10M 1 to about 1011 M 1. Antibodies may, for example, bind with an EC50 of 50 nM or less, 10 nM or less, 1 nM or less, 100 pM or less, or more preferably 10 pM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the anti-Cx43 hemichannel blocking antibodies described herein can specifically bind to their intended target. In some aspects, the anti-Cx43 hemichannel blocking antibodies described herein have no off site binding. For example, the anti-Cx43 hemichannel blocking antibodies described herein bind only to their intended target at a particular target site and do not bind or are not distributed to the heart, liver or spinal cord.

The anti-Cx43 hemichannel blocking antibodies described herein can be variants including, without limitation, a fragment (e.g., an Fab fragment or an F(ab′)2 fragment of, e.g., a tetrameric antibody), a fragment of an scFv or diabody, or a variant of a tetrameric antibody, an scFv, a diabody, or fragments thereof that differ by virtue of the addition and/or substitution of one or more amino acid residues. The anti-Cx43 hemichannel blocking antibody moiety can be further engineered as, for example, a di-diabody.

As is well known in the art, certain types of antibody fragments can be generated by enzymatic treatment of a “full-length” antibody. Digestion with papain produces two identical Fab fragments, each with a single antigen-binding site, and a residual Fc fragment. The Fab fragment also contains the constant domain of the light chain and the Chi domain of the heavy chain. In contrast, digestion with pepsin yields the F(ab′)2 fragment that has two antigen-binding sites and is still capable of cross-linking antigen.

Fab′ fragments differ from Fab fragments in that they include additional residues at the C-terminus of the Chi domain, including one or more cysteine residues from the antibody hinge region. The cysteine residues of the constant domains bear a free thiol group. F(ab′)2 antibody fragments are pairs of Fab′ fragments linked by cysteine residues in the hinge region. Other chemical couplings of antibody fragments are also known in the art.

The Fv region is a minimal fragment that contains a complete antigen-recognition and binding site consisting of one heavy chain and one light chain variable domain. The three CDRs of each variable domain interact to define an antigen-biding site on the surface of the VH-VL dimer. Collectively, the six CDRs confer antigen-binding specificity to the antibody.

A “single-chain variable fragment (scFv)” means a protein comprising the variable regions of the heavy and light chains of an antibody. As would be known in the art, a “single-chain” antibody or “scFv” fragment is a single chain Fv variant formed when the VH and VL domains of an antibody are included in a single polypeptide chain that recognizes and binds an antigen. Typically, single-chain antibodies include a polypeptide linker between the VH and VL domains that allows the scFv to form a desired three-dimensional structure for antigen binding (see, e.g., Pluckthun, In The Pharmacology of Monoclonal Antibodies, Rosenburg and Moore Eds., Springer-Verlag, New York, 113:269-315. 1994). For example, a scFv can be a fusion protein comprising a variable heavy chain, a linker, and a variable light chain. In some aspects, the linker can be a short, flexible fragment that can be about 8 to 20 amino acids in length. For example, (G4S)n can be used (n=1, 2, 3 or 4).

In some aspects, the anti-Cx43 hemichannel blocking antibody can be a diabody. Diabodies are small antibody fragments that have two antigen-binding sites. Each fragment contains a VH domain concatenated to a VL domain. However, since the linker between the domains is too short to allow pairing between them on the same chain, the linked Vh-Vl domains are forced to pair with complementary domains of another chain, creating two antigen-binding sites. Diabodies are described more fully, for example, in EP 404,097; WO 93/11161; and Hollinger et al., Proc. Natl. Acad. Sci. USA 90:6444-6448, 1993.

In some aspects, an anti-Cx43 hemichannel blocking antibody or a fragment thereof that binds to at least a portion of Cx43 protein and inhibits or blocks the opening of Cx43 hemichannels and is associated with protecting visual function, reducing retinal ganglion cell loss, reduces retinal ganglion apoptosis, protects or preserves retinal ganglion cell axon, somata and dendrites, reduces reactive gliosis, reduces microglia activation, and reduces neuroinflammation in the retina are contemplated. In some aspects, an anti-Cx43 hemichannel blocking antibody or a fragment thereof that binds to at least a portion of Cx43 protein and inhibits or blocks the opening of Cx43 hemichannels and protecting visual function, reducing retinal ganglion cell loss, reduces retinal ganglion apoptosis, protects or preserves retinal ganglion cell axon, somata and dendrites, reduces reactive gliosis, reduces microglia activation, and reduces neuroinflammation in the retina are contemplated. In some aspects, the anti-Cx43 hemichannel blocking antibody can be a monoclonal antibody, polyclonal antibody or a humanized antibody. Thus, by known means and as described herein, polyclonal or monoclonal antibodies, antibody fragments, and binding domains and CDRs (including engineered forms of any of the foregoing) may be created that are specific to Cx43 protein, one or more of its respective epitopes, or conjugates of any of the foregoing, whether such antigens or epitopes are isolated from natural sources or are synthetic derivatives or variants of the natural compounds.

Examples of anti-Cx43 hemichannel blocking antibody fragments suitable include without limitation: (i) the Fab fragment, consisting of VL, VH, CL, and CH1 domains; (ii) the “Fd” fragment consisting of the VII and Cm domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VII domain and a VL domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the VH and VL domains. Minibodies comprising a scFv joined to a CH3 domain may also be made (Hu et al., 1996).

Antibody-like binding peptidomimetics are also contemplated. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a Cx43 extracellular domain protein, in order to produce antibodies specific for Cx43 protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256:495-497, 1975), or by recombinant DNA methods. A monoclonal antibody is a single species of antibody wherein every antibody molecule recognizes the same epitope because the antibody producing cells are derived from a single B-lymphocyte cell line. The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a Cx43 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells may be isolated from freshly prepared rabbit peripheral blood mononuclear cells of immunized rabbits and further selected for Cx43 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. A Cx43 specific binding Fab may be selected out through multiple rounds enrichment panning and sequenced. Selected Cx43 binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In some aspects, the anti-Cx43 hemichannel blocking antibody can be a chimeric antibody, for example, an antibody comprising antigen binding sequences from a non-human donor grafted to a heterologous non-human, human, or humanized sequence (e.g., framework and/or constant domain sequences). The term “chimeric antibody” (or “chimeric immunoglobulin”) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al. (1984), infra; Morrison et al., Proc. Natl. Acad. Sci. U.S.A. 81:6851).

Methods have been developed to replace light and heavy chain constant domains of the monoclonal antibody with analogous domains of human origin, leaving the variable regions of the foreign antibody intact. Alternatively, “fully human” monoclonal antibodies can be produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992); Barbas et al. (1994); and Schier et al. (1996).

It is fully expected that anti-Cx43 hemichannel blocking antibodies will have the ability to neutralize or counteract the effects of Cx43 regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described herein, antibodies may also be constructed so as to be chimeric or partially or fully human, so as to reduce or eliminate the adverse immunological consequences resulting from administering to an animal an antibody that has been produced in, or has sequences from, other species.

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds Cx43.

An anti-Cx43 hemichannel blocking antibody or preferably an immunological portion of an anti-Cx43 hemichannel blocking antibody, can be chemically conjugated to, or expressed as, a fusion protein with other proteins. For purposes of this specification and the accompanying claims, all such fused proteins are included in the definition of antibodies or an immunological portion of an antibody.

Described herein are antibodies (e.g., anti-Cx43 hemichannel blocking antibodies) and antibody-like molecules against Cx43, polypeptides and peptides that are linked to at least one agent to form an antibody conjugate or payload. In order to increase the efficacy of antibody molecules as diagnostic or therapeutic agents, to the antibody can be linked or covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In some aspects, the anti-Cx43 hemichannel blocking antibodies described herein can comprise a heavy chain immunoglobulin variable region comprising complementarity determining region 1 (CDR1) comprising the sequence of SEQ ID NO: 19; CDR2 comprising the sequence of SEQ ID NO: 20; and a CDR3 comprising the sequence of SEQ ID NO: 21. Table 3 shows examples of CDRs of heavy chains of anti-Cx43 hemichannel blocking antibodies.

In some aspects, the anti-Cx43 hemichannel blocking antibodies described herein can comprise a light chain immunoglobulin variable region comprising complementarity determining region 1 (CDR1) comprising the sequence of SEQ ID NO: 31; CDR2 comprising the sequence of SEQ ID NO: 32; and a CDR3 comprising the sequence of SEQ ID NO: 33. Table 3 shows examples of CDRs in light chains of anti-Cx43 hemichannel blocking antibodies.

In some aspects, the anti-Cx43 hemichannel blocking antibodies described herein can comprise a heavy chain immunoglobulin variable region comprising complementarity determining region 1 (CDR1) comprising the sequence of SEQ ID NO: 19; CDR2 comprising the sequence of SEQ ID NO: 20; and a CDR3 comprising the sequence of SEQ ID NO: 21; and a light chain immunoglobulin variable region comprising complementarity determining region 1 (CDR1) comprising the sequence of SEQ ID NO: 31; CDR2 comprising the sequence of SEQ ID NO: 32; and a CDR3 comprising the sequence of SEQ ID NO: 33.

In some aspects, the anti-Cx43 hemichannel blocking antibodies described herein can comprise a variable heavy chain comprising a sequence having at least 90% identity to a sequence set forth in SEQ ID NOs: 19, 20, or 21 (see, Table 3). In some aspects, the anti-Cx43 hemichannel blocking antibodies described herein comprises a variable heavy chain comprising a sequence having at least 90, 91, 92, 93, 94, 95, 96, 97, 98, 99, or 100% identity to a sequence set forth in SEQ ID NOs: 19, 20 or 21.

In some aspects, the anti-Cx43 hemichannel blocking antibodies described herein can comprise a variable light chain comprising a sequence having at least 90% identity to a sequence set forth in SEQ ID NOs: 31, 32 or 33 (see, Table 3). In some aspects, the anti-Cx43 hemichannel blocking antibodies described herein comprises a variable light chain comprising a sequence having at least 90, 91, 92, 93, 94, 95, 96, 97, 98, 99, or 100% identity to a sequence set forth in SEQ ID NOs: 31, 32 or 33.

Disclosed herein are nucleic acid sequences that encode MIH comprising the sequence of SEQ ID NO: 52. Disclosed herein are nucleic acid sequences that encode MIKI comprising the sequence of SEQ ID NO: 54.

Disclosed herein are nucleic acid sequences encoding MIH comprising a variable heavy chain comprising a sequence having at least 90% identity to a sequence set forth in SEQ ID NO: 52 (see, Table 4). In some aspects, MIH comprises a variable heavy chain comprising a sequence having at least 90, 91, 92, 93, 94, 95, 96, 97, 98, 99 or 100% identity to a sequence set forth in SEQ ID NO: 52.

Disclosed herein are nucleic acid sequences encoding MIKI comprising a variable light chain comprising a sequence having at least 90% identity to a sequence set forth in SEQ ID NO: 54 (see, Table 4). In some aspects, MIKI comprises a variable light chain comprising a sequence having at least 90, 91, 92, 93, 94, 95, 96, 97, 98, 99 or 100% identity to a sequence set forth in SEQ ID NO: 54.

Disclosed herein are nucleic acid sequences that encode the MIH region comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18.

Disclosed herein are nucleic acid sequences that encode the MIKI region comprising a light chain immunoglobulin variable region comprising a CDR1 comprising the sequence of SEQ ID NO: 28; a CDR2 comprising a comprising the sequence of SEQ ID NO: 29; a CDR3 comprising a comprising the sequence of SEQ ID NO: 30.

Disclosed herein are nucleic acid sequences that encode anti-Cx43 hemichannel blocking antibodies comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18; and a light chain immunoglobulin variable region comprising a CDR1 comprising the sequence of SEQ ID NO: 28; a CDR2 comprising a comprising the sequence of SEQ ID NO: 29; a CDR3 comprising a comprising the sequence of SEQ ID NO: 30.

Disclosed herein are anti-Cx43 hemichannel blocking antibodies or fragments thereof that bind to human Cx43. Also described herein are anti-Cx43 hemichannel blocking antibodies. In some aspects, the anti-Cx43 hemichannel blocking antibodies or fragments thereof comprises a variable heavy chain comprising a sequence having at least 90% identity to one of the variable heavy chain amino acid sequences provided in Tables 3 or 5. In some aspects, the anti-Cx43 hemichannel blocking antibodies or fragments thereof comprises a variable heavy chain comprising a sequence having at least 90% identity to a sequence set forth in SEQ ID NO: 58. In some aspects, the anti-Cx43 hemichannel blocking antibodies or fragments thereof comprises a variable heavy chain comprising a sequence set forth in SEQ ID NO: 58.

Disclosed herein are anti-Cx43 antibodies or fragments thereof that bind to human Cx43. Also described herein are anti-Cx43 hemichannel blocking antibodies. In some aspects, the anti-Cx43 hemichannel blocking antibodies or fragments thereof comprises a variable light chain comprising a sequence having at least 90% identity to one of the variable light chain amino acid sequences provided in Tables 3 or 5. In some aspects, the anti-Cx43 hemichannel blocking antibodies or fragments thereof comprises a variable light chain comprising a sequence having at least 90% identity to a sequence set forth in SEQ ID NO: 60. In some aspects, the anti-Cx43 hemichannel blocking antibodies or fragments thereof comprises a variable light chain comprising a sequence set forth in SEQ ID NOs: 60.

Disclosed herein are anti-Cx43 antibodies or fragments thereof that bind to human Cx-43 hemichannels. Also described herein are anti-Cx43 hemichannel blocking antibodies. In some aspects, the anti-Cx43 hemichannel blocking antibodies or fragments thereof comprises a variable heavy chain comprising a sequence having at least 90% identity to a sequence set forth in SEQ ID NO: 58, and a variable light chain comprising a sequence having at least 90% identity to a sequence set forth in SEQ ID NO: 60. In some aspects, the anti-Cx43 hemichannel blocking antibody comprises a variable heavy chain comprising a sequence set forth in SEQ ID NO: 58 and a variable light chain comprising a sequence set forth in SEQ ID NO: 60.

In some aspects, the anti-Cx43 hemichannel blocking antibodies or fragments thereof comprises a MIH region. In some aspects, the MIH region comprises a heavy chain immunoglobulin variable region comprising a CDR1 comprising the sequence of SEQ ID NO: 19; a CDR2 comprising the sequence of SEQ ID NO: 20; and a CDR3 comprising the sequence of SEQ ID NO: 21.

In some aspects, the anti-Cx43 hemichannel blocking antibodies or fragments thereof comprises a MIKI region. In some aspects, the MIKI region comprises a light chain immunoglobulin variable region comprising a CDR1 comprising the sequence of SEQ ID NO: 31; a CDR2 comprising the sequence of 32; and a CDR3 comprising the sequence of SEQ ID NO: 33.

In some instances, the disclosed anti-Cx43 hemichannel blocking antibodies or fragments thereof further comprise a tag sequence.

Disclosed herein are nucleic acid sequences that encode the disclosed antibodies (e.g., anti-Cx43 hemichannel blocking antibodies) or fragments thereof. For example, disclosed are nucleic acid sequences comprising a variable heavy chain comprising a sequence having at least 90% identity to a sequence set forth in SEQ ID NO: 52. Disclosed herein are nucleic acid sequences that encode the disclosed anti-Cx43 antibodies hemichannel blocking or fragments thereof. For example, disclosed herein are nucleic acid sequences comprising a variable heavy chain comprising a sequence set forth in SEQ ID NO: 52. Also disclosed herein are nucleic acid sequences comprising a variable light chain comprising a sequence having at least 90% identity to a sequence set forth in SEQ ID NO: 54. Also disclosed are nucleic acid sequences comprising a variable light chain comprising a sequence set forth in SEQ ID NO: 54.

Disclosed herein are nucleic acid sequences comprising a variable heavy chain comprising a sequence having at least 90% identity to a sequence set forth in SEQ ID NO: 52; and a variable light chain comprising a sequence having at least 90% identity a sequence set forth in SEQ ID NO: 54. Disclosed are nucleic acid sequences comprising a variable heavy chain comprising a sequence set forth in SEQ ID NO: 52; and a variable light chain comprising a sequence set forth in SEQ ID NO: 54.

Disclosed herein are nucleic acid sequences capable of encoding a single chain variable fragment comprising a variable heavy chain comprising a sequence having at least 90% identity a sequence set forth in SEQ ID NO: 52.

Disclosed are nucleic acid sequences capable of encoding a single chain variable fragment comprising a variable light chain comprising a sequence having at least 90% identity a sequence set forth in SEQ ID NO: 54.

Disclosed are nucleic acid sequences capable of encoding a single chain variable fragment comprising a variable heavy chain comprising a sequence having at least 90% identity a sequence set forth in SEQ ID NO:58; and a variable light chain comprising a sequence having at least 90% identity a sequence set forth in SEQ ID NO:60.

In some instances, the disclosed anti-Cx43 hemichannel blocking antibodies or fragments thereof can be bispecific. For example, the anti-Cx43 hemichannel blocking antibody or fragment thereof can comprise a first Fab region comprising the heavy and light chain of SEQ ID NO: 58 and a second Fab region comprising the heavy and light chain of SEQ ID NO: 60, wherein the first and second Fab regions can be different.

In some instances, the bispecific anti-Cx43 hemichannel blocking antibodies can be trifunctional.

In some instances, the disclosed anti-Cx43 hemichannel blocking antibodies or fragments thereof can be mouse, human, humanized, chimeric, or a combination thereof.

In some instances, the disclosed anti-Cx43 hemichannel blocking antibodies or fragments thereof are monoclonal.

### Methods

Disclosed herein are methods of treating or preventing glaucoma in a subject comprising administering to the subject a therapeutically effective amount of an anti-connexin 43 antibody or a fragment thereof. Disclosed herein are methods of treating or preventing macular degeneration in a subject with anti-Cx43 hemichannel blocking antibodies or fragments thereof. Disclosed herein are methods of treating or preventing an eye disease or an eye injury in a subject with anti-Cx43 hemichannel blocking antibodies or fragments thereof. Disclosed herein are methods of treating or preventing diabetic retinopathy in a subject with anti-Cx43 hemichannel blocking antibodies or fragments thereof. Disclosed herein are methods of reducing or ameliorating one or more symptoms of glaucoma in a subject with anti-Cx43 hemichannel blocking antibodies or fragments thereof. Disclosed herein are methods of reducing or ameliorating one or more symptoms of macular degeneration in a subject with anti-Cx43 hemichannel blocking antibodies or fragments thereof. Disclosed herein are methods of reducing or ameliorating one or more symptoms of an eye disease or an eye injury in a subject with anti-Cx43 hemichannel blocking antibodies or fragments thereof. Disclosed herein are methods of reducing or ameliorating one or more symptoms of diabetic retinopathy in a subject with anti-Cx43 hemichannel blocking antibodies or fragments thereof. Disclosed herein are methods of treating or preventing retinal injury in a subject with anti-Cx43 hemichannel blocking antibodies or fragments thereof. Disclosed herein are methods of treating or preventing optical nerve damage or degeneration in a subject with anti-Cx43 hemichannel blocking antibodies or fragments thereof. Disclosed herein are methods of reducing or ameliorating one or more symptoms of retinal injury in a subject with anti-Cx43 hemichannel blocking antibodies or fragments thereof. Disclosed herein are methods of reducing or ameliorating one or more symptoms of optical nerve damage or degeneration in a subject with anti-Cx43 hemichannel blocking antibodies or fragments thereof. Disclosed herein are methods of protecting visual function in a subject with anti-Cx43 hemichannel blocking antibodies or fragments thereof. Disclosed herein are methods of reducing retinal ganglion cell loss or reducing retinal ganglion apoptosis in a subject with anti-Cx43 hemichannel blocking antibodies or fragments thereof. Disclosed herein are methods of protecting or preserving retinal ganglion cell axon, somata or dendrites in a subject with anti-Cx43 hemichannel blocking antibodies or fragments thereof. Disclosed herein are methods of reducing reactive gliosis in a subject with anti-Cx43 hemichannel blocking antibodies or fragments thereof. Disclosed herein are methods of reducing microglia activation or neuroinflammation in the retina in a subject with anti-Cx43 hemichannel blocking antibodies or fragments thereof. Disclosed herein are methods of reducing optical nerve damage and degeneration in a subject with anti-Cx43 hemichannel blocking antibodies or fragments thereof.

In some aspects, the anti-Cx43 hemichannel blocking antibody or fragment thereof can comprise a variable heavy chain comprising a sequence having at least 90% identity to a sequence set forth in SEQ ID NO: 58. In some aspects, the anti-Cx43 hemichannel blocking antibody or fragment thereof can comprise a variable light chain comprising a sequence having at least 90% identity to a sequence set forth in SEQ ID NO: 60. In some aspects, the anti-Cx43 hemichannel blocking antibody or fragment thereof can comprise a variable heavy chain comprising a sequence having at least 90% identity to a sequence set forth in SEQ ID NO: 58 and a variable light chain comprising a sequence having at least 90% identity to a sequence set forth in SEQ ID NO: 60.

In some aspects, the anti-Cx43 hemichannel blocking antibody or fragment thereof can comprise: a heavy chain immunoglobulin variable region comprising: a first complementarity determining region 1 comprising a sequence having at least 60% identity to SEQ ID NO: 19; a second complementarity determining region 2 comprising a sequence having at least 60% identity to SEQ ID NO: 20; and a third complementarity determining region 3 comprising a sequence having at least 60% identity to SEQ ID NO: 21. In some aspects, the anti-Cx43 hemichannel blocking antibody or fragment thereof can comprise: a light chain immunoglobulin variable region comprising: a first complementarity determining region 1 comprising a sequence having at least 60% identity to SEQ ID NO: 31; a second complementarity determining region 2 comprising a sequence having at least 60% identity to SEQ ID NO: 32; and a third complementarity determining region 3 comprising a sequence having at least 60% identity to SEQ ID NO: 33.

In some aspects, the anti-Cx43 hemichannel blocking antibody or fragment thereof can comprise: a heavy chain immunoglobulin variable region comprising: a first complementarity determining region 1 comprising a sequence having a single amino acid change compared to SEQ ID NO: 19; a second complementarity determining region 2 comprising a sequence a single amino acid change compared to SEQ ID NO: 20; and a third complementarity determining region 3 comprising a sequence a single amino acid change compared to SEQ ID NO: 21. In some aspects, the anti-Cx43 hemichannel blocking antibody or fragment thereof can comprise: a light chain immunoglobulin variable determining region comprising: a first complementarity determining region 1 comprising a sequence having a single amino acid change compared to SEQ ID NO: 31; a second complementarity determining region 2 comprising a sequence having a single amino acid change compared to SEQ ID NO: 32; and a third complementarity determining region 3 comprising a sequence having a single amino acid change compared to SEQ ID NO: 33.

In some aspects, the anti-Cx43 hemichannel blocking antibody or fragment thereof can comprise a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof (e.g., SEQ ID NO: 67); and/or c) a complementarity determining region (CDR3) comprising the sequence of SEQ ID NO: 21 or a variant thereof. In some aspects, any one of the heavy chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the at least one amino acid substitution can decrease or reduce deamidation.

In some aspects, the anti-Cx43 hemichannel blocking antibody or fragment thereof can comprise a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 or a variant thereof. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation.

In some aspects, the anti-Cx43 hemichannel blocking antibody or fragment thereof can comprise a heavy chain immunoglobulin variable region comprising: a complementarity determining region 1 (CDR1) comprising a sequence having at least 60% identity to a sequence set forth in SEQ ID NO: 19; a complementarity determining region 2 (CDR2) comprising a sequence having at least 60% identity to a sequence set forth in SEQ ID NO: 20; and/or a complementarity determining region 3 (CDR3) comprising a sequence having at least 60% identity to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation. For example, the at least one amino acid substitution can be a glycine residue to an alanine residue. In some aspects, the antibody or fragment thereof can comprise a heavy chain immunoglobulin variable region comprising a CDR2 comprising a sequence set forth in SEQ ID NO: 67 (INPSNAGT).

In some aspects, the anti-Cx43 hemichannel blocking antibody or fragment thereof can comprise a light chain immunoglobulin variable region comprising: a complementarity determining region 1 (CDR1) comprising a sequence having at least 60% identity to a sequence set forth in SEQ ID NO: 31; a complementarity determining region 2 (CDR2) comprising a sequence having at least 60% identity to a sequence set forth in SEQ ID NO: 32; and/or a complementarity determining region 3 (CDR3) comprising a sequence having at least 60% identity to a sequence set forth in SEQ ID NO: 33. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation.

In some aspects, the anti-Cx43 hemichannel blocking antibody or fragment thereof can comprise a heavy chain immunoglobulin variable region comprising: a complementarity determining region 1 (CDR1) comprising a sequence having a single amino acid change compared to a sequence set forth in SEQ ID NO: 19; a complementarity determining region 2 (CDR2) comprising a sequence having a single amino acid change compared to a sequence set forth in SEQ ID NO: 20; and/or a complementarity determining region 3 (CDR3) comprising a sequence having a single amino acid change compared to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation. For example, the at least one amino acid substitution can be a glycine residue to an alanine residue. In some aspects, the antibody or fragment thereof can comprise a heavy chain immunoglobulin variable region comprising a CDR2 comprising a sequence set forth in SEQ ID NO: 67 (INPSNAGT).

In some aspects, the anti-Cx43 hemichannel blocking antibody or fragment thereof can comprise a light chain immunoglobulin variable region comprising: a complementarity determining region 1 (CDR1) comprising a sequence having a single amino acid change compared to a sequence set forth in SEQ ID NO: 31; a complementarity determining region 2 (CDR2) comprising a sequence having a single amino acid change compared to a sequence set forth in SEQ ID NO: 32; and/or a complementarity determining region 3 (CDR3) comprising a sequence having a single amino acid change compared to a sequence set forth in SEQ ID NO: 33. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid. In some aspects, the other amino acid can be an alanine. In some aspects, the at least one amino acid substitution can decrease or reduce deamidation.

In some aspects, any of the methods disclosed herein can comprise administering to the subject an effective amount of an expression vector encoding the anti-Cx43 hemichannel blocking antibody or fragment thereof. In some aspects, the anti-Cx43 hemichannel blocking antibody or fragment thereof can be administered in a pharmaceutically acceptable composition. In some aspects, the pharmaceutical composition can be lyophilized. In some aspects, the anti-Cx43 hemichannel blocking antibody or fragment thereof can be administered systemically. In some aspects, the anti-Cx43 hemichannel blocking antibody or fragment thereof can be administered intravenously, subconjunctivally, intraocularly, intra-vitreally or locally. In some aspects, the anti-Cx43 hemichannel blocking antibody or fragment thereof can be a humanized antibody or humanized fragment thereof. In some aspects, the anti-Cx43 hemichannel blocking antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20 (or SEQ ID NO: 67), a third VH CDR corresponding to SEQ ID NO: 21, a first VL CDR corresponding to SEQ ID NO: 31, a second VL CDR corresponding to SEQ ID NO: 32, and a third VL CDR corresponding to SEQ ID NO: 33. In some aspects, the antibody can be an IgG class of antibody, wherein the IgG class antibody is an IgG1, IgG2, IgG3, or IgG4 class antibody.

Disclosed herein are methods of treating or preventing glaucoma in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing macular degeneration in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing an eye disease or an eye injury in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing diabetic retinopathy in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing retinal injury in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing optical nerve damage or degeneration in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody hemichannel blocking or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing retinal ganglion cell loss or reducing retinal ganglion apoptosis in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of protecting or preserving retinal ganglion cell axon, somata or dendrites in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing reactive gliosis in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the a anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing microglia activation or neuroinflammation in the retina in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing optical nerve damage and degeneration in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of protecting visual function in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing or ameliorating one or more symptoms of glaucoma, macular degeneration, an eye disease or an eye injury, diabetic retinopathy, retinal injury or optical nerve damage or degeneration in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of inhibiting the opening of a connextin 43 hemichannel in an astrocyte in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the opening of the Cx hemichannel is induced by an inflammatory factor. In some aspects, the inflammatory factor can be interleukin-1beta/TNG-alpha. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of protecting retinal ganglion cell (RGC) dendritic damage in a subject, the methods comprising administering to the subject a therapeutically effective amount of an anti-connexin 43 antibody or a fragment thereof. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the RGC dendritic damage can be caused by an inflammatory cytokine. In some aspects, the inflammatory cytokine can be IL01beta/TNF-alpha. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

In some aspects, the methods can comprise administering to the subject a therapeutically effective amount of an anti-connexin 43 hemichannel blocking antibody (or fragment thereof. In some aspects, the anti-Cx43 hemichannel blocking antibody or fragment thereof can comprise a variable heavy chain comprising a sequence having at least 90% identity to a sequence set forth in SEQ ID NO: 58. In some aspects, the anti-Cx43 hemichannel blocking antibody or fragment thereof can comprise a variable light chain comprising a sequence having at least 90% identity to a sequence set forth in SEQ ID NO: 60. In some aspects, the anti-Cx43 hemichannel blocking antibody or fragment thereof can comprise a variable heavy chain comprising a sequence having at least 90% identity to a sequence set forth in SEQ ID NO: 58 and a variable light chain comprising a sequence having at least 90% identity to a sequence set forth in SEQ ID NO: 60.

In some aspects, the methods can reduce retinal ganglion cell loss or reducing retinal ganglion apoptosis. In some aspects, the methods can protect or preserve retinal ganglion cell axon, somata or dendrites. In some aspects, the methods can reduce reactive gliosis. In some aspects, the methods can reduce microglia activation or neuroinflammation in the retina. In some aspects, the methods can reduce optical nerve damage and degeneration in a subject. In some aspects, the methods can reduce or ameliorate one or more symptoms of glaucoma, macular degeneration, an eye disease or an eye injury, diabetic retinopathy, retinal injury or optical nerve damage or degeneration.

In some aspects, the one or more symptoms of glaucoma can be loss of side or peripheral vision, seeing halos around lights, redness in the eye, an eye that looks hazy (particularly in infants), upset stomach or vomiting, eye pain or any combination thereof. In some aspects, the glaucoma can be open-angle glaucoma. In some aspects, glaucoma that is the open-angle type may not present with any symptoms or may be asymptomatic. Examples of symptoms of open-angle glaucoma can include but are not limited to reduced vision and loss of peripheral vision, swollen or bulging cornea, pupil dilation (that does not change with increasing or decreasing light), redness in the while of the eye, and nausea. In some aspects, the glaucoma can be angle-closure glaucoma. Examples of symptoms of be angle-closure glaucoma can include but are not limited headache, eye pain, nausea and vomiting, blurred vision, halos around lights, and eye redness.

In some aspects, the one or more symptoms of macular degeneration can be blurry or fuzzy vision, difficulty recognizing familiar faces, straight lines appear wavy, a dark, empty area or blind spot appearing in the center of vision, loss of central vision or a combination thereof. Examples of symptoms of macular degeneration also include but are not limited to gradual or sudden changes in the quality of vision (e.g., straight lines appearing in a subject's vision), changes (fast or slow) in central vision, dark, blurry areas or whiteout that appears in the center of vision, and a change in the perception of color.

In some aspects, the eye disease can be associated or compromised with/by a reduced hydraulic conductivity and metabolic exchange via Bruch's membrane, diseases characterized by an accumulation of extra-/intracellular lipids in the eye, diseases that use lipid-derived mediators of inflammation or benefit from oxidized lipid removal as well as diseases that benefit from Bruch's membrane remodeling. In some aspects, the eye disease can be macular degeneration, age related maculopathy (ARM), age related macular degeneration (AMD) including both the dry and wet forms of age related macular degeneration, glaucoma, ocular hypertension, macular edema, retinal pigment epithelium detachments, coats disease, uveitis, sicca syndrome, hereditary diseases associated with increased extra-/intracellular lipid storage/accumulation, and juvenile macular degeneration. In some aspects, the eye injury can be traumatic glaucoma. In some aspects, the one or more symptoms of an eye disease or an eye injury can refer to reduction, prevention, or elimination of one or more symptoms characteristic of eye disease and/or associated pathologies. Such a reduction includes, but is not limited to a reduction or elimination of oxidized phospholipids, accumulation of extracellular lipids in Bruch's membranes, accumulation of lipid rich debris in Bruch's membranes, vision loss, formation of choriocapillaris, thickening of the Bruch's membrane, accumulation of neutral lipids in the Bruch's membrane, formation of a diffusion barrier between the retinal pigment epithelium, deposition of debris (basal linear deposits and drusen) between the basal membrane of the RPE and the inner collagenous layer, accumulation of lipofuscin in the RPE cells, RPE atrophy, photoreceptor degeneration, choroidal neovascularization, trapped fluid accumulation in the retina and retinal pigment epithelial cells, elevated intraocular pressure, as well as leakage, bleeding, scarring of the eye, and the like. Examples of symptoms of an eye disease or an eye injury include but are not limited to accumulation of extracellular lipids in Bruch's membranes, accumulation of lipid rich debris, vision loss, formation of choriocapillaris, thickening of the Bruch's membrane, accumulation of neutral lipids in the Bruch's membrane, formation of a diffusion barrier between the retinal pigment epithelium and choriocapillaris, deposition of debris (basal linear deposits and drusen) between the basal membrane of the RPE, and the inner collagenous layer, accumulation of lipofuscin in the RPE cells, RPE atrophy, photoreceptor degeneration, choroidal neovascularization, as well as leakage, bleeding, scarring of the eye.

Age-related macular degeneration can sometimes begin with characteristic yellow deposits in the macula (central area of the retina which provides detailed central vision) called drusen between the retinal pigment epithelium and the underlying choroid. Most people with these early changes (sometimes referred to as age-related maculopathy) have good vision. People with drusen can go on to develop advanced macular degeneration. The risk is considerably higher when the drusen are large and numerous and associated with disturbance in the pigmented cell layer under the macula. In some aspects, large and soft drusen can be related to elevated cholesterol deposits and may respond to cholesterol lowering agents or the Rheo Procedure.

Advanced AMD, which is responsible for profound vision loss, has two forms: dry and wet. Central geographic atrophy, the dry form of advanced AMD, results from atrophy to the retinal pigment epithelial layer below the retina, which causes vision loss through loss of photoreceptors (rods and cones) in the central part of the eye. In some aspects, vitamin supplements along with high doses of antioxidants, lutein and zeaxanthin, can be co-administered with the anti-connexin 43 antibody or a fragment thereof disclosed herein to slow the progression of dry macular degeneration, and in some patients, improve visual acuity.

Neovascular or exudative AMD, the wet form of advanced AMD, causes vision loss due to abnormal blood vessel growth in the choriocapillaries, through Bruch's membrane, ultimately leading to blood and protein leakage below the macula. Bleeding, leaking, and scarring from these blood vessels eventually cause irreversible damage to the photoreceptors and rapid vision loss if left untreated. Examples of agents that can be co-administered with the anti-connexin 43 antibody or a fragment thereof disclosed herein for treating wet macular degeneration included but are not limited to anti-angiogenics or anti-VEGF (anti-Vascular Endothelial Growth Factor) agents, when injected directly into the vitreous humor of the eye using a small, painless needle, can cause regression of the abnormal blood vessels and improvement of vision. In some aspects, these injections can be repeated on a monthly or bi-monthly basis. Examples of these agents include Lucentis, Avastin and Macugen.

AMD: dry from: geographic atrophy. The remodeling of Bruch's membrane provides an undisturbed passage between retinal pigment epithelium and choriocapillaris, which is important for the health of the retina. The retinal pigment epithelium stands with the choriocapillaris in a close relationship and they are dependent on each other. An uncompromised communication between these structures improves the blood supply for the outer retina by the choriocapillaris and the retinal pigment epithelium layer integrity by improved anchorage on Bruch's membrane via water soluble proteins.

AMD: wet form: choroidal neovascularization. The same mechanism as for dry AMD applies for the wet form. Due to the improved environmental conditions, retinal pigment epithelium cells also reduce the secretion of pro-angiogenic factors, which normally keeps a neovascularization active for a longer period. In combination with anti-angiogenic treatments pro-angiogenic mechanisms are not just temporarily blocked but the secretion stimulus can be long-term reduced.

A characteristic of glaucoma/ocular hypertension is elevated intraocular pressure (IOP). The treatment of the age-related “lipid wall” in Bruch's membrane increases the hydraulic conductivity along Bruch's membrane again and facilitates fluid transport from the vitreous to the choroid (vitreoretinal-choroidal outflow, uveoscleral outflow), which can normalize the IOP.

Macular edema, Retinal pigment epithelium detachments. Macular edema is characterized by trapped fluid accumulations in the retina and RPE detachments by fluid accumulations under the retinal pigment epithelium. The normal fluid transport is directed across Bruch's membrane into the choriocapillaris.

In some aspects, the one or more symptoms of diabetic retinopathy can be the appearance of floaters (e.g., tiny specks that seem to drift through the field of vision), flashes of light in one or both eyes (photopsia), blurred vision or a combination thereof. Examples of symptoms of diabetic retinopathy include but are not limited to pain, trouble seeing, cuts to the eyelid, one eye not moving as well, one eye sticks out, blood in the clear part of the eye, unusual pupil size or shape, and something embedded in the eye.

In some aspects, the one or more symptoms of retinal injury can be retinal detachment. Examples of symptoms of retinal injury include but are not limited to the appearance of floaters (e.g., tiny specks that seem to drift through the field of vision), flashes of light in one or both eyes (photopsia), blurred vision or a combination thereof. In some aspects, the one or more symptoms of retinal injury can be traumatic glaucoma. Traumatic glaucoma can be any glaucoma caused by an injury to the eye. This type of glaucoma can occur both immediately after an injury to the eye or years later. In some aspects, retinal injury can be caused by an injury that “bruises” the eye (e.g., called blunt trauma) or injuries that penetrate the eye. In some aspects, the antibodies (e.g., M1 antibody disclosed herein can be administered with antibiotics or corticosteroid therapy to help prevent, reduce or treat tissue (e.g., retinal tissue) damage and scarring.

In some aspects, the one or more symptoms of optical nerve damage or degeneration can be optic neuritis, pain, vision loss (including temporary vision loss), optic neuropathy, optic nerve atrophy or a combination thereof. Optic neuritis occurs when swelling (inflammation) damages the optic nerve. The optic nerve is a bundle of nerve fibers that transmits visual information from the eye to the brain. Examples of symptoms of optic neuritis include but are not limited to pain with eye movement and temporary vision loss in one eye. Optic neuropathy, which can take the form of non-arteritic anterior ischemic optic neuropathy (NAION), can damage the optic nerve as a result of a change in blood flow. When blood flow to the optic nerve is interrupted, it doesn't receive the oxygen it needs. Optic nerve atrophy is damage to the optic nerve. Optic nerve atrophy can be caused by poor blood flow to the eye, disease, trauma, or exposure to toxic substances.

In some aspects, the anti-Cx43 hemichannel blocking antibody or fragment thereof can comprise: a heavy chain immunoglobulin variable region comprising: a first complementarity determining region 1 comprising a sequence having at least 60% identity to SEQ ID NO: 19; a second complementarity determining region 2 comprising a sequence having at least 60% identity to SEQ ID NO: 20; and a third complementarity determining region 3 comprising a sequence having at least 60% identity to SEQ ID NO: 21. In some aspects, the anti-Cx43 hemichannel blocking antibody or fragment thereof can comprise: a light chain immunoglobulin variable region comprising: a first complementarity determining region 1 comprising a sequence having at least 60% identity to SEQ ID NO: 31; a second complementarity determining region 2 comprising a sequence having at least 60% identity to SEQ ID NO: 32; and a third complementarity determining region 3 comprising a sequence having at least 60% identity to SEQ ID NO: 33.

In some aspects, the anti-Cx43 hemichannel blocking antibody or fragment thereof can comprise: a heavy chain immunoglobulin variable region comprising: a first complementarity determining region 1 comprising a sequence having a single amino acid change compared to SEQ ID NO: 19; a second complementarity determining region 2 comprising a sequence a single amino acid change compared to SEQ ID NO: 20; and a third complementarity determining region 3 comprising a sequence a single amino acid change compared to SEQ ID NO: 21. In some aspects, the anti-Cx43 hemichannel blocking antibody or fragment thereof can comprise: a light chain immunoglobulin variable determining region comprising: a first complementarity determining region 1 comprising a sequence having a single amino acid change compared to SEQ ID NO: 31; a second complementarity determining region 2 comprising a sequence having a single amino acid change compared to SEQ ID NO: 32; and a third complementarity determining region 3 comprising a sequence having a single amino acid change compared to SEQ ID NO: 33.

In some aspects, the anti-Cx43 hemichannel blocking antibody or fragment thereof can comprise a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, any one of the heavy chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects the anti-Cx43 hemichannel blocking antibody or fragment thereof can comprise a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 or a variant thereof. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 hemichannel blocking antibody or fragment thereof can comprise a heavy chain immunoglobulin variable region comprising: a complementarity determining region 1 (CDR1) comprising a sequence having at least 60% identity to a sequence set forth in SEQ ID NO: 19; a complementarity determining region 2 (CDR2) comprising a sequence having at least 60% identity to a sequence set forth in SEQ ID NO: 20; and/or a complementarity determining region 3 (CDR3) comprising a sequence having at least 60% identity to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 hemichannel blocking antibody or fragment thereof can comprise a light chain immunoglobulin variable region comprising: a complementarity determining region 1 (CDR1) comprising a sequence having at least 60% identity to a sequence set forth in SEQ ID NO: 31; a complementarity determining region 2 (CDR2) comprising a sequence having at least 60% identity to a sequence set forth in SEQ ID NO: 32; and/or a complementarity determining region 3 (CDR3) comprising a sequence having at least 60% identity to a sequence set forth in SEQ ID NO: 33. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 hemichannel blocking antibody or fragment thereof can comprise a heavy chain immunoglobulin variable region comprising: a complementarity determining region 1 (CDR1) comprising a sequence having a single amino acid change compared to a sequence set forth in SEQ ID NO: 19; a complementarity determining region 2 (CDR2) comprising a sequence having a single amino acid change compared to a sequence set forth in SEQ ID NO: 20; and/or a complementarity determining region 3 (CDR3) comprising a sequence having a single amino acid change compared to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 hemichannel blocking antibody or fragment thereof can comprise a light chain immunoglobulin variable region comprising: a complementarity determining region 1 (CDR1) comprising a sequence having a single amino acid change compared to a sequence set forth in SEQ ID NO: 31; a complementarity determining region 2 (CDR2) comprising a sequence having a single amino acid change compared to a sequence set forth in SEQ ID NO: 32; and/or a complementarity determining region 3 (CDR3) comprising a sequence having a single amino acid change compared to a sequence set forth in SEQ ID NO: 33. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 hemichannel blocking antibody or a fragment thereof, wherein the anti-connexin 43 hemichannel blocking antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the anti-connexin 43 hemichannel blocking antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or SEQ ID NO: 67, or a variant thereof; and/or c) a complementarity determining region (CDR3) comprising the sequence of SEQ ID NO: 21 or a variant thereof. In some aspects, the anti-connexin 43 hemichannel blocking antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the at least one amino acid substitution can decrease or reduce deamidation. In some aspects, the anti-connexin 43 hemichannel blocking antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the anti-connexin 43 hemichannel blocking antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the anti-connexin 43 hemichannel blocking antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the anti-connexin 43 hemichannel blocking antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the anti-connexin 43 hemichannel blocking antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

In some aspects, any of the methods disclosed herein can comprise administering to the subject an effective amount of an expression vector encoding an anti-Cx43 hemichannel blocking antibody or fragment thereof. In some aspects, the anti-Cx43 hemichannel blocking antibody or fragment thereof can be administered in a pharmaceutically acceptable composition. In some aspects, the pharmaceutical composition can be lyophilized. In some aspects, the anti-Cx43 hemichannel blocking antibody or fragment thereof can be administered systemically. In some aspects, the anti-Cx43 hemichannel blocking antibody or fragment thereof can be administered intravenously, intradermally, intratumorally, intramuscularly, intraperitoneally, subcutaneously, or locally. In some aspects, the anti-Cx43 hemichannel blocking antibody or fragment thereof can be a humanized antibody or humanized fragment thereof. In some aspects, the anti-Cx43 hemichannel blocking antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 31, a second VL CDR corresponding to SEQ ID NO: 32, and a third VL CDR corresponding to SEQ ID NO: 33. In some aspects, the antibody can be an IgG class of antibody, wherein the IgG class antibody is an IgG1, IgG2, IgG3, or IgG4 class antibody.

In some aspects, any of the methods disclosed herein can further comprise administering at least a second therapeutic agent or second therapy to the subject. In some aspects, the second therapeutic agent can be a nonsteroidal anti-inflammatory drug (NSAID), vitamin B12 or a combination thereof. In some aspects, a second therapeutic agent and a second therapy can be administered to the subject.

In some aspects, in any of the methods disclosed herein the anti-Cx43 hemichannel blocking antibody or fragment thereof can bind to a Cx43 hemichannel. In some aspects, in any of the methods disclosed herein the anti-Cx43 hemichannel antibody or fragment thereof can inhibit or block the opening of a Cx43 hemichannel.

In some aspects, the anti-Cx43 hemichannel blocking antibody or fragment thereof can further comprise a tag sequence.

In some aspects, the anti-Cx43 hemichannel blocking antibody or fragment thereof can be a Fab fragment an Fab′ fragment or an F(ab′)2 fragment.

In some aspects, the anti-Cx43 hemichannel blocking antibody useful in any of the methods disclosed herein can be an anti-Cx43 hemichannel blocking antibody comprising one or more of the sequences set forth in Table 1.

TABLE 1
Examples of sequences of anti-Cx43 hemichannel
blocking antibodies.

| SEQ | | |
|---|---|---|
| ID NO: | SEQUENCE | NAME |
| 68 | EVQLVQSGAEVKKPGASVKVSCKASGYTFTSYYM | Heavy |
| | YWVRQAPGQGLEWIGGINPSNAGTNFNEKFKNRA | chain |
| | TLTVDKSTSTAYMELSSLRSEDTAVYYCTREGNP | variable |
| | YYTMNYWGQGTLVTVSS | domain |
| 69 | DIVMTQSPDSLAVSLGERATISCKSSQSLLNSGN | Light |
| | QKTYLAWYQQKPGQPPKLLIYGASTRESGVPDRF | chain |
| | SGSGSGTDFTLTISSLQAEDVAVYYCQNDHSYPF | variable |
| | TFGQGTKLEIK | domain |
| 70 | EVQLVQSGAEVKKPGASVKVSCKASGYTFTSYYM | Heavy |
| | YWVRQAPGQGLEWIGGINPSNgGTNFNEKFKNRA | chain |
| | TLTVDKSTSTAYMELSSLRSEDTAVYYCTREGNP | (variable |
| | YYTMNYWGQGTLVTVSSASTKGPSVFPLAPSSKS | region and |
| | TSGGTAALGCLVKDYFPEPVTVSWNSGALTSGVH | constant |
| | TFPAVLQSSGLYSLSSVVTVPSSSLGTQTYICNV | region) |
| | NHKPSNTKVDKKVEPKSCDKTHTCPPCPAPELLG | |
| | GPSVFLFPPKPKDTLMISRTPEVTCVVVDVSHED | |
| | PEVKFNWYVDGVEVHNAKTKPREEQYNSTYRVVS | |
| | VLTVLHQDWLNGKEYKCKVSNKALPAPIEKTISK | |
| | AKGQPREPQVYTLPPSRDELTKNQVSLTCLVKGF | |
| | YPSDIAVEWESNGQPENNYKTTPPVLDSDGSFFL | |
| | YSKLTVDKSRWQQGNVFSCSVMHEALHNHYTQKS | |
| | LSLSPGK | |
| 71 | EVQLVQSGAEVKKPGASVKVSCKASGYTFTSYYM | Heavy |
| | YWVRQAPGQGLEWIGGINPSNgGTNFNEKFKNRA | chain |
| | TLTVDKSTSTAYMELSSLRSEDTAVYYCTREGNP | (variable |
| | YYTMNYWGQGTLVTVSSASTKGPSVFPLAPCSRS | region and |
| | TSESTAALGCLVKDYFPEPVTVSWNSGALTSGVH | constant |
| | TFPAVLQSSGLYSLSSVVTVPSSSLGTKTYTCNV | region) |
| | DHKPSNTKVDKRVESKYGPPCPPCPAPEFLGGPS | |
| | VFLFPPKPKDTLMISRTPEVTCVVVDVSQEDPEV | |
| | QFNWYVDGVEVHNAKTKPREEQFNSTYRVVSVLT | |
| | VLHQDWLNGKEYKCKVNKGLPSSIEKTISKAKGQ | |
| | PREPQVYTLPPSQEEMTKNQVSLTCLVKGFYPSD | |
| | IAVEWESNGQPENNYKTTPPVLDSDGSFFLYSRL | |
| | TVDKSRWQEGNVFSCSVMHEALHNHYTQKSLSLS | |
| | LGK | |
| 72 | EVQLVQSGAEVKKPGASVKVSCKASGYTFTSYYM | Heavy |
| | YWVRQAPGQGLEWIGGINPSNaGTNFNEKFKNRA | chain |
| | TLTVDKSTSTAYMELSSLRSEDTAVYYCTREGNP | (variable |
| | YYTMNYWGQGTLVTVSSASTKGPSVFPLAPCSRS | region and |
| | TSESTAALGCLVKDYFPEPVTVSWNSGALTSGVH | constant |
| | TFPAVLQSSGLYSLSSVVTVPSSSLGTKTYTCNV | region) |
| | DHKPSNTKVDKRVESKYGPPCPPCPAPEFLGGPS | |
| | VFLFPPKPKDTLMISRTPEVTCVVVDVSQEDPEV | |
| | QFNWYVDGVEVHNAKTKPREEQFNSTYRVVSVLT | |
| | VLHQDWLNGKEYKCKVSNKGLPSSIEKTISKAKG | |
| | QPREPQVYTLPPSQEEMTKNQVSLTCLVKGFYPS | |
| | DIAVEWESNGQPENNYKTTPPVLDSDGSFFLYSR | |
| | LTVDKSRWQEGNVFSCSVMHEALHNHYTQKSLSL | |
| | SLGK | |
| 73 | EVQLVQSGAEVKKPGASVKVSCKASGYTFTSYYM | Heavy |
| | YWVRQAPGQGLEWIGGINPSNgGTNFNEKFKNRA | chain |
| | TLTVDKSTSTAYMELSSLRSEDTAVYYCTREGNP | (variable |
| | YYTMNYWGQGTLVTVSSASTKGPSVFPLAPSSKS | region and |
| | TSGGTAALGCLVKDYFPEPVTVSWNSGALTSGVH | constant |
| | TFPAVLQSSGLYSLSSVVTVPSSSLGTQTYICNV | region) |
| | NHKPSNTKVDKKVEPKSCDKTHTCPPCPAPEAAG | |
| | GPSVFLFPPKPKDTLMISRTPEVTCVVVDVSHED | |
| | PEVKFNWYVDGVEVHNAKTKPREEQYNSTYRVVS | |
| | VLTVLHQDWLNGKEYKCKVSNKALPAPIEKTISK | |
| | AKGQPREPQVYTLPPSRDELTKNQVSLTCLVKGF | |
| | YPSDIAVEWESNGQPENNYKTTPPVLDSDGSFFL | |
| | YSKLTVDKSRWQQGNVFSCSVMHEALHNHYTQKS | |
| | LSLSPGK | |
| 74 | EVQLVQSGAEVKKPGASVKVSCKASGYTFTSYYM | Heavy |
| | YWVRQAPGQGLEWIGGINPSNaGTNFNEKFKNRA | chain |
| | TLTVDKSTSTAYMELSSLRSEDTAVYYCTREGNP | (variable |
| | YYTMNYWGQGTLVTVSSASTKGPSVFPLAPSSKS | region and |
| | TSGGTAALGCLVKDYFPEPVTVSWNSGALTSGVH | constant |
| | TFPAVLQSSGLYSLSSVVTVPSSSLGTQTYICNV | region) |
| | NHKPSNTKVDKKVEPKSCDKTHTCPPCPAPEAAG | |
| | GPSVFLFPPKPKDTLMISRTPEVTCVVVDVSHED | |
| | PEVKFNWYVDGVEVHNAKTKPREEQYNSTYRVVS | |
| | VLTVLHQDWLNGKEYKCKVSNKALPAPIEKTISK | |
| | AKGQPREPQVYTLPPSRDELTKNQVSLTCLVKGF | |
| | YPSDIAVEWESNGQPENNYKTTPPVLDSDGSFFL | |
| | YSKLTVDKSRWQQGNVFSCSVMHEALHNHYTQKS | |
| | LSLSPGK | |
| 75 | EVQLVQSGAEVKKPGASVKVSCKASGYTFTSYYM | Heavy |
| | YWVRQAPGQGLEWIGGINPSNgGTNFNEKFKNRA | chain |
| | TLTVDKSTSTAYMELSSLRSEDTAVYYCTREGNP | (variable |
| | YYTMNYWGQGTLVTVSSASTKGPSVFPLAPCSRS | region and |
| | TSESTAALGCLVKDYFPEPVTVSWNSGALTSGVH | constant |
| | TFPAVLQSSGLYSLSSVVTVPSSSLGTKTYTCNV | region) |
| | DHKPSNTKVDKRVESKYGPPCPPCPAPEFEGGPS | |
| | VFLFPPKPKDTLMISRTPEVTCVVVDVSQEDPEV | |
| | QFNWYVDGVEVHNAKTKPREEQFNSTYRVVSVLT | |
| | VLHQDWLNGKEYKCKVSNKGLPSSIEKTISKAKG | |
| | QPREPQVYTLPPSQEEMTKNQVSLTCLVKGFYPS | |
| | DIAVEWESNGQPENNYKTTPPVLDSDGSFFLYSR | |
| | LTVDKSRWQEGNVFSCSVMHEALHNHYTQKSLSL | |
| | SLGK | |
| 76 | EVQLVQSGAEVKKPGASVKVSCKASGYTFTSYYM | Heavy |
| | YWVRQAPGQGLEWIGGINPSNaGTNFNEKFKNRA | chain |
| | TLTVDKSTSTAYMELSSLRSEDTAVYYCTREGNP | (variable |
| | YYTMNYWGQGTLVTVSSASTKGPSVFPLAPCSRS | region and |
| | TSESTAALGCLVKDYFPEPVTVSWNSGALTSGVH | constant |
| | TFPAVLQSSGLYSLSSVVTVPSSSLGTKTYTCNV | region) |
| | DHKPSNTKVDKRVESKYGPPCPPCPAPEFEGGPS | |
| | VFLFPPKPKDTLMISRTPEVTCVVVDVSQEDPEV | |
| | QFNWYVDGVEVHNAKTKPREEQFNSTYRVVSVLT | |
| | VLHQDWLNGKEYKCKVSNKGLPSSIEKTISKAKG | |
| | QPREPQVYTLPPSQEEMTKNQVSLTCLVKGFYPS | |
| | DIAVEWESNGQPENNYKTTPPVLDSDGSFFLYSR | |
| | LTVDKSRWQEGNVFSCSVMHEALHNHYTQKSLSL | |
| | SLGK | |
| 77 | EVQLVQSGAEVKKPGASVKVSCKASGYTFTSYYM | Heavy |
| | YWVRQAPGQGLEWIGGINPSNgGTNFNEKFKNRA | chain |
| | TLTVDKSTSTAYMELSSLRSEDTAVYYCTREGNP | (variable |
| | YYTMNYWGQGTLVTVSSASTKGPSVFPLAPCSRS | region and |
| | TSESTAALGCLVKDYFPEPVTVSWNSGALTSGVH | constant |
| | TFPAVLQSSGLYSLSSVVTVPSSSLGTKTYTCNV | region) |
| | DHKPSNTKVDKRVESKYGPPCPPCPAPEAAGGPS | |
| | VFLFPPKPKDTLMISRTPEVTCVVVDVSQEDPEV | |
| | QFNWYVDGVEVHNAKTKPREEQFNSTYRVVSVLT | |
| | VLHQDWLNGKEYKCKVSNKGLPSSIEKTISKAKG | |
| | QPREPQVYTLPPSQEEMTKNQVSLTCLVKGFYPS | |
| | DIAVEWESNGQPENNYKTTPPVLDSDGSFFLYSR | |
| | LTVDKSRWQEGNVFSCSVMHEALHNHYTQKSLSL | |
| | SLGK | |
| 78 | EVQLVQSGAEVKKPGASVKVSCKASGYTFTSYYM | Heavy |
| | YWVRQAPGQGLEWIGGINPSNaGTNFNEKFKNRA | chain |
| | TLTVDKSTSTAYMELSSLRSEDTAVYYCTREGNP | (variable |
| | YYTMNYWGQGTLVTVSSASTKGPSVFPLAPCSRS | region and |
| | TSESTAALGCLVKDYFPEPVTVSWNSGALTSGVH | constant |
| | TFPAVLQSSGLYSLSSVVTVPSSSLGTKTYTCNV | region) |
| | DHKPSNTKVDKRVESKYGPPCPPCPAPEAAGGPS | |
| | VFLFPPKPKDTLMISRTPEVTCVVVDVSQEDPEV | |
| | QFNWYVDGVEVHNAKTKPREEQFNSTYRVVSVLT | |
| | VLHQDWLNGKEYKCKVSNKGLPSSIEKTISKAKG | |
| | QPREPQVYTLPPSQEEMTKNQVSLTCLVKGFYPS | |
| | DIAVEWESNGQPENNYKTTPPVLDSDGSFFLYSR | |
| | LTVDKSRWQEGNVFSCSVMHEALHNHYTQKSLSL | |
| | SLGK | |
| 79 | DIVMTQSPDSLAVSLGERATISCKSSQSLLNSGN | Light |
| | QKTYLAWYQQKPGQPPKLLIYGASTRESGVPDRF | chain |
| | SGSGSGTDFTLTISSLQAEDVAVYYCQNDHSYPF | (variable |
| | TFGQGTKLEIKRTVAAPSVFIFPPSDEQLKSGTA | region and |
| | SVVCLLNNFYPREAKVQWKVDNALQSGNSQESVT | constant |
| | EQDSKDSTYSLSSTLTLSKADYEKHKVYACEVTH | region) |
| | QGLSSPVTKSFNRGEC | |

### Treatment of Diseases

Disclosed herein are anti-Cx43 hemichannel blocking antibodies (e.g., the M1 antibody) and biological fragments thereof that can be used to treat or prevent glaucoma, macular degeneration, diabetic retinopathy, retina injury, or optical nerve damage and degeneration in a subject. Also disclosed herein are anti-Cx43 hemichannel blocking antibodies and biological fragments thereof that can be used to treat or prevent eye disease or eye injuries in a subject. In some aspects, the anti-Cx43 hemichannel blocking antibodies or biological fragments thereof can be used to protect visual function, reduce retinal ganglion cell loss, reduce retinal ganglion apoptosis, protect or preserve retinal ganglion cell axon, somata and dendrites, reduce reactive gliosis, reduce microglia activation, or reduce neuroinflammation in the retina in a subject in need thereof.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with an eye disease or an eye injury, glaucoma, macular degeneration, diabetic retinopathy, retina injury, or optical nerve damage and degeneration or one or more symptoms of eye disease or an eye injury, glaucoma, macular degeneration, diabetic retinopathy, retina injury, or optical nerve damage and degeneration in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of eye disease or an eye injury, glaucoma, macular degeneration, diabetic retinopathy, retina injury, or optical nerve damage and degeneration may involve, for example, protection of visual function, a reduction of retinal ganglion cell loss, a reduction of retinal ganglion apoptosis, protection or preservation of retinal ganglion cell axon, somata and dendrites, a reduction of reactive gliosis, a reduction in microglia activation, or a reduction in neuroinflammation in the retina.

In some aspects, the eye disease can be associated or compromised with/by a reduced hydraulic conductivity and metabolic exchange via Bruch's membrane, diseases characterized by an accumulation of extra-/intracellular lipids in the eye, diseases that use lipid-derived mediators of inflammation or benefit from oxidized lipid removal as well as diseases that benefit from Bruch's membrane remodeling.

In some aspects, the eye disease can be macular degeneration, age related maculopathy (ARM), age related macular degeneration (AMD) including both the dry and wet forms of age related macular degeneration, glaucoma, ocular hypertension, macular edema, retinal pigment epithelium detachments, coats disease, uveitis, sicca syndrome, hereditary diseases associated with increased extra-/intracellular lipid storage/accumulation, and juvenile macular degeneration.

In some aspects, the eye injury can be traumatic glaucoma or retinal detachment. In some aspects, the retinal detachment can be caused by a severe eye trauma.

In some aspects, the one or more symptoms of an eye disease or an eye injury can refer to reduction, prevention, or elimination of one or more symptoms characteristic of eye disease and/or associated pathologies. Such a reduction includes, but is not limited to a reduction or elimination of oxidized phospholipids, accumulation of extracellular lipids in Bruch's membranes, accumulation of lipid rich debris in Bruch's membranes, vision loss, formation of choriocapillaris, thickening of the Bruch's membrane, accumulation of neutral lipids in the Bruch's membrane, formation of a diffusion barrier between the retinal pigment epithelium, deposition of debris (basal linear deposits and drusen) between the basal membrane of the RPE and the inner collagenous layer, accumulation of lipofuscin in the RPE cells, RPE atrophy, photoreceptor degeneration, choroidal neovascularization, trapped fluid accumulation in the retina and retinal pigment epithelial cells, elevated intraocular pressure, as well as leakage, bleeding, scarring of the eye, and the like.

Disclosed herein, are methods of treating a patient with an eye disease or an eye injury, glaucoma, macular degeneration, diabetic retinopathy, retina injury, or optical nerve damage and degeneration or preventing blindness, microglia activation, neuroinflammation, optical nerve damage or optical nerve degeneration. Also disclosed herein, are methods of treating or preventing an eye disease or an eye injury in a subject. In some aspects, the subject has been diagnosed with an eye disease or an eye injury, glaucoma, macular degeneration, diabetic retinopathy, retina injury, or optical nerve damage and degeneration prior to the administering step.

In some aspects, the methods can comprise administering an effective amount of the anti-Cx43 hemichannel blocking antibody to the subject. In some aspects, the method can comprise administering an effective amount of an expression vector encoding the anti-Cx43 hemichannel blocking antibody to the subject. In some aspects, the anti-Cx43 hemichannel blocking antibody that binds to a connexin 43 (Cx43) hemichannel and blocks or inhibits channel opening.

The compositions described herein can be formulated to include a therapeutically effective amount of the anti-Cx43 hemichannel blocking antibodies or fragments thereof disclosed herein. In some aspects, anti-Cx43 hemichannel blocking antibodies or fragments thereof disclosed herein can be contained within a pharmaceutical formulation. In some aspects, the pharmaceutical formulation can be a unit dosage formulation.

The therapeutically effective amount or dosage of any of the anti-Cx43 hemichannel blocking antibodies or fragments thereof used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the disease symptoms), the age and physical characteristics of the subject and other considerations known to those of ordinary skill in the art. Dosages can be established using clinical approaches known to one of ordinary skill in the art. A therapeutically effective dosage of an anti-Cx43 hemichannel blocking antibody can result in a decrease in severity of one or more disease symptoms, an increase in frequency and duration of disease symptom-free periods, or a prevention of impairment or disability due to the disease affliction. A therapeutically effective amount of a therapeutic compound or antibody can protect visual function, reduce retinal ganglion cell loss, reduce retinal ganglion apoptosis, protect or preserve retinal ganglion cell axon, somata and dendrites, reduce reactive gliosis, reduce microglia activation, and reduce neuroinflammation in the retina, or otherwise ameliorate symptoms in a subject.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the anti-Cx43 hemichannel blocking antibodies or compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The anti-Cx43 hemichannel blocking antibodies, fragments thereof or compositions described herein can be administered in conjunction with other therapeutic modalities to a subject in need of therapy. The present compounds can be given to prior to, simultaneously with or after treatment with other agents or regimes. For example, the anti-Cx43 hemichannel blocking antibodies disclosed herein can be administered alone or in conjunction with standard therapies used to treat any of the eye diseases disclosed herein, including but not limited to glaucoma, macular degeneration, diabetic retinopathy, retina injury, and optical nerve damage and degeneration. In some aspects, any of the anti-Cx43 hemichannel blocking antibodies or compositions described herein can be administered or used together with vitamin B12, corticosteroid, antibiotics or a combination thereof.

### Pharmaceutical Compositions

Disclosed herein are compositions, e.g., pharmaceutical compositions, comprising one or a combination of anti-Cx43 hemichannel blocking antibodies, or antigen-binding portion(s), or fragments thereof formulated with a pharmaceutically acceptable carrier. Such compositions may include one or a combination of (e.g., two or more different) antibodies, antibody fragments or immunoconjugates described herein. For example, a pharmaceutical composition described herein can comprise a combination of antibodies or antibody fragments thereof that bind to different epitopes on the target antigen or that have complementary activities.

Disclosed herein are pharmaceutical compositions comprising any of the anti-Cx43 hemichannel blocking antibodies or fragments thereof as described herein with a pharmaceutically acceptable carrier. Also disclosed herein are anti-Cx43 hemichannel blocking antibodies or pharmaceutical compositions for use as a medicament or for use in therapy for treating or preventing an eye disease, an eye injury, glaucoma, macular degeneration, diabetic retinopathy, retina injury, optical nerve damage and degeneration, protecting visual function, reducing retinal ganglion cell loss, reducing retinal ganglion apoptosis, protecting or preserving retinal ganglion cell axon, somata and dendrites, reducing reactive gliosis, reducing microglia activation, or reducing neuroinflammation in the retina.

Pharmaceutical compositions of the invention also can be administered as combination therapy, i.e., combined with other agents. For example, the combination therapy can include an anti-hemichannel antibody combined with at least one other therapeutic agent or therapy.

As used herein, the phrase “pharmaceutically acceptable carrier” includes any solvents, dispersion media, coatings, antibacterial and antifungal agents, isotonic and absorption delaying agents, and the like that are physiologically compatible. Preferably, the carrier can be suitable for intravenous, intramuscular, subcutaneous, or parenteral administration (e.g., by injection or infusion). Depending on the route of administration, the active compound, i.e., antibody, or immunoconjugate, may be coated in a material to protect the compound from the action of acids and other natural conditions that may inactivate the compound.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated herein, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the anti-Cx43 hemichannel blocking antibody, the dosage ranges from about 0.0001 to 100 mg/kg, and more usually 0.01 to 5 mg/kg, 5 mg/kg to 10 mg/kg, 10 mg/kg to 15 mg/kg, 15 mg/kg to 20 mg/kg or 20 mg/kg to 25 mg/kg of the host body weight. In some aspects, the dosages can be 0.3 mg/kg body weight, 1 mg/kg body weight, 3 mg/kg body weight, 5 mg/kg body weight or 10 mg/kg body weight or within the range of 1-10 mg/kg. In some aspects, the dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, 25 mg/kg body weight or 30 mg/kg body weight or within the range of 1-30 mg/kg. In some aspects, the dosages can be about 15, 16, 17, 18, 19, 20, 21, 22, 23, 24 or 25 mg/kg body weight. In some aspects, the dosages can be 5 mg/kg body weight. In some aspects, the dosages can be 15 mg/kg body weight. In some aspects, the dosages can be 20 mg/kg body weight. In some aspects, the dosages can be 25 mg/kg body weight. An exemplary treatment regime entails administration once per week, once every two weeks, once every three weeks, once every four weeks, once a month, once every 3 months or once every three to 6 months. Preferred dosage regimens for an anti-hemichannel antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more anti-Cx43 hemichannel blocking antibodies with different binding specificities are administered simultaneously, in which case the dosage of each anti-Cx43 hemichannel blocking antibody administered falls within the ranges indicated. The anti-Cx43 hemichannel blocking antibody is usually administered on multiple occasions. Intervals between single dosages can be, for example, weekly, monthly, every three months or yearly. Intervals can also be irregular as indicated by measuring blood levels of the anti-Cx43 hemichannel blocking antibody to the target antigen in the patient. In some methods, dosage is adjusted to achieve a plasma anti-Cx43 hemichannel blocking antibody concentration of about 1-1000 μg/ml and in some methods about 25-300 μg/ml.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions disclosed herein employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Any of the compositions disclosed herein can be administered via one or more routes of administration using one or more of a variety of methods known in the art. As will be appreciated by the skilled artisan, the route and/or mode of administration will vary depending upon the desired results. In some aspects, the routes of administration for antibodies disclosed herein include but are not limited to intravenous, intramuscular, intradermal, intraperitoneal, subcutaneous, or other parenteral routes of administration, for example by injection or infusion. The phrase “parenteral administration” as used herein means modes of administration other than enteral and topical administration, usually by injection, and includes, without limitation, intravenous, intramuscular, intraarterial, intraperitoneal, transtracheal, subcutaneous, subcuticular, intraarticular injection and infusion.

In some aspects, the routes of administration for antibodies disclosed herein include but are not limited to oral administration, sublingual administration, trans-buccal mucosa administration, transdermal administration, administration by inhalation, nasal administration, topical administration, intravaginal administration, ophthalmic administration, intraaural administration, intracerebral administration, intrathecal administration, rectal administration, intraperitoneal administration, and parenteral administration, including injectable such as intravenous administration, intra-arterial administration, intramuscular administration, intradermal administration, and subcutaneous administration. Ophthalmic administration can include topical administration, subconjunctival administration, sub-Tenon's administration, epibulbar administration, retrobulbar administration, intra-orbital administration, and intraocular administration, which includes intra-vitreal administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

In some aspects, the anti-Cx43 hemichannel blocking antibodies disclosed herein can be administered directly to the eye. This route of administration, known as ophthalmic administration can be accomplished by a variety of means known to a person of skill in the art. For example, and not to be limiting, the antibodies can be provided to an eye of a subject in need thereof through the placement of a cream, an ointment, or a liquid drop preparation onto the inner eyelid of the subject, through the use of a mist sprayed onto the eye of the subject, or through intravitreal injection. Ophthalmic administration can further include topical administration, subconjunctival administration, sub-Tenon's administration, epibulbar administration, retrobulbar administration, intra-orbital administration, periocular injection, and intraocular administration, which includes intra-vitreal administration.

In some aspects, ophthalmic administration of the anti-Cx43 hemichannel blocking antibodies disclosed herein can be accomplished through the use of systemic delivery, such intravenous delivery, unidirectional episcleral implant, hollow microneedles, solid coated microneedles, free-floating intravitreal implant, or scleral-fixated intravitreal implant. Ophthalmic administration of the anti-Cx43 hemichannel blocking antibodies can also be accomplished through the use of topical iontophoresis. Iontophoresis is a noninvasive method of delivering compounds into the eye. It can be performed by applying a small electrical current that has the same charge as the compound to create repulsive electromotive forces that enable delivery of the compound to the anterior or posterior segment of the eye. Edelhauser et al., Ophthalmic Drug Delivery Systems for the Treatment of Retinal Diseases: Basic Research to Clinical Applications, IOVS 2010: 51 (11) 5403-5419, which is herein incorporated in its entirety by this reference, describes these various methods of ophthalmic administration.

Combination Treatments. The compositions and methods described herein can involve an anti-Cx43 hemichannel blocking antibody or an antibody fragment thereof against Cx43 to inhibit or block the opening of the Cx43 hemichannel to, for example, protect visual function, reduce retinal ganglion cell loss, reduce retinal ganglion apoptosis, protect or preserve retinal ganglion cell axon, somata and dendrites, reduce reactive gliosis, reduce microglia activation, and reduce neuroinflammation in the retina, reduce or ameliorate one or more symptoms of an eye disease, an eye injury, glaucoma, macular degeneration, diabetic retinopathy, retina injury, or optical nerve damage and degeneration, in combination with a second or additional therapeutic agent or therapy. Such therapy can be applied in the treatment of any disease that is associated with Cx43-mediated retinal ganglion cell loss, retinal ganglion apoptosis, injury or degeneration to retinal ganglion cell axon, somata and dendrites, reactive gliosis, microglia activation, or neuroinflammation in the retina. For example, the disease can be any eye disease or eye injury disclosed herein, glaucoma, macular degeneration, diabetic retinopathy, retina injury, or optical nerve damage and degeneration.

The methods and compositions, including combination therapies, enhance the therapeutic or protective effect, and/or increase the therapeutic effect of another anti-inflammatory agent, therapeutic agent or therapy. Therapeutic and prophylactic methods and compositions can be provided in a combined amount effective to achieve the desired effect, such as protecting visual function, reducing retinal ganglion cell loss, reducing retinal ganglion apoptosis, protecting or preserving retinal ganglion cell axon, somata and dendrites, reducing reactive gliosis, reducing microglia activation, and reducing neuroinflammation in the retina and/or reducing or ameliorating one or more symptoms of an eye disease or an eye injury, glaucoma, macular degeneration, diabetic retinopathy, retina injury, or optical nerve damage and degeneration. This process may involve contacting the cells with both an anti-Cx43 hemichannel blocking antibody or antibody fragment and a second therapy. A tissue, tumor, or cell can be contacted with one or more compositions or pharmacological formulation(s) comprising one or more of the agents (i.e., anti-Cx43 hemichannel blocking antibody or antibody fragment or a second therapeutic agent), or by contacting the tissue, tumor, and/or cell with two or more distinct compositions or formulations, wherein one composition provides 1) an anti-Cx43 hemichannel blocking antibody or antibody fragment, 2) a second therapeutic agent, or 3) both an anti-Cx43 hemichannel blocking antibody or antibody fragment and a second therapeutic agent. Also, it is contemplated that such a combination therapy can be used in conjunction with surgical therapy, for example.

The terms “contacted” and “exposed,” when applied to a cell, are used herein to describe the process by which a therapeutic construct and a second therapeutic agent are delivered to a target cell or are placed in direct juxtaposition with the target cell. To achieve the desired outcome, for example, both agents can be delivered to a cell in a combined amount effective to the desired outcome.

The anti-Cx43 hemichannel blocking antibodies or fragments thereof can be administered before, during, after, or in various combinations relative to a second therapeutic agent or therapy. The administrations may be in intervals ranging from concurrently to minutes to days to weeks. In aspects where the anti-Cx43 hemichannel blocking antibody or antibody fragment is provided to a patient separately from a second therapeutic agent or therapy, one would generally ensure that a significant period of time did not expire between the time of each delivery, such that the two compounds would still be able to exert an advantageously combined effect on the patient. In such instances, it is contemplated that one may provide a patient with the anti-Cx43 hemichannel blocking antibody therapy and the second therapeutic agent or therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no a second therapeutic agent or therapy is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some aspects, the anti-Cx43 hemichannel blocking antibodies or fragments thereof disclosed herein can be administered one or more times separated by one or more days. In some aspects, a second, third, fourth, fifth, and so on, administration of the anti-Cx43 hemichannel blocking antibodies or fragments thereof disclosed herein can be separated by 1, 2, 3, 4, 5, 6, or 7 days. In some aspects, the anti-Cx43 hemichannel blocking antibodies or fragments thereof disclosed herein can be administered one or more times separated by one or more weeks. In some aspects, a second, third, fourth, fifth, and so on, administration of the anti-Cx43 antibodies or fragments thereof disclosed herein can be separated by 1, 2, 3, 4, 5, 6, 7, 8, 9, 10, 11, or 12 weeks. In some aspects, the anti-Cx43 hemichannel blocking antibodies or fragments thereof disclosed herein can be administered one or more times separated by one or more months. In some aspects, a second, third, fourth, fifth, and so on, administration of the anti-Cx43 antibodies or fragments thereof disclosed herein can be separated by 1, 2, 3, 4, 5, 6, 7, 8, 9, 10, 11 or 12 months. In some aspects, the anti-Cx43 hemichannel blocking antibodies or fragments thereof disclosed herein can be administered one or more times separated by a variety of intervals, days, weeks, months or any combination thereof.

Various combinations may be employed. For the example below an antibody therapy is “A” and a second therapeutic agent or therapy is “B”:

A/B/A B/A/B B/B/A A/A/B A/B/B B/A/A A/B/B/B B/A/B/B B/B/B/A B/B/A/B
A/A/B/B A/B/A/B A/B/B/A B/B/A/A B/A/B/ A B/A/A/B A/A/A/B B/A/A/A A/B/A/A
A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

### Kits and Diagnostics

Disclosed herein are kits comprising one or more therapeutic agents and/or other therapeutic and delivery agents. In some aspects, the kit can be used for preparing and/or administering a therapy disclosed herein. The kit may comprise one or more sealed vials containing any of the pharmaceutical compositions disclosed herein. The kit may include, for example, at least one anti-Cx43 hemichannel blocking antibody or fragment thereof as well as reagents to prepare, formulate, and/or administer the components one or more of the compositions disclosed herein or perform one or more steps of the inventive methods. In some aspects, the kit may also comprise a suitable container, which can be a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

Application No. PCT/US2017/019605 (WO 2017-147561) provides examples of anti-Cx43 antibodies, CDR sequences, heavy chain and light chain sequences, nucleic acid sequences that encode said antibodies and the epitope sequences that said antibodies bind that can be used in the disclosed methods, which is hereby incorporated by reference for teaching the same.

Anti-Cx43 antibodies were generated and clones were identified that produced Cx43-binding antibodies. Disclosed herein are anti-Cx43 antibodies comprising CDR sequences shown in the Tables below along with the pairing for each of the characterized antibodies. Examples of both DNA and amino acids for anti-Cx43 antibody sequences are also shown in the Tables below along with the correct pairing for each of the characterized antibodies. Examples of anti-Cx43 antibodies, include, but are not limited to M1 and M2 antibodies. The M1 antibody (an anti-connexin 43 hemichannel blocking antibody) inhibits or blocks the opening of a Cx43 hemichannel. The M2 antibody activates, stimulates and/or enhances the opening of a Cx43 hemichannel.

As used herein, “M1 antibody” refers to an antibody comprising a variable heavy chain comprising a sequence having the sequence set forth in SEQ ID NO: 58; and a variable light chain comprising the sequence set forth in SEQ ID NO: 60.

As used herein, “M2 antibody” refers to an antibody comprising a variable heavy chain comprising a sequence having the sequence set forth in SEQ ID NO: 58; and a variable light chain comprising the sequence set forth in SEQ ID NO: 63.

Also disclosed are humanized anti-Cx43 antibodies. For example, disclosed herein are humanized M1 and M2 antibodies.

TABLE 2
Pairing of heavy chain and light chain for
two functional anti-Cx43 antibodies.

| Antibody Name | Heavy chain | Light chain |
|---|---|---|
| M1 (HmAb1) | M1H | M1K1 or M1K1a |
| M2 (HmAb2) | M1H | M1M7K |

TABLE 3
Sequence of antibody chains of anti-Cx43 antibodies from the hybridomas
generated.

| mAb | CDR-1 | CDR-2 | CDR-3 |
|---|---|---|---|
| M1H | ggctacaccttcaccagcta | attaatcctagcaatggtggt | acaagagagggtaaccccta |
| | ctat | act | ctatactatgaactac |
| | (SEQ ID NO: 16) | (SEQ ID NO: 17 | (SEQ ID NO: 18) |
| | GYTFTSYY | INPSNGGT | TREGNPYYTMNY |
| | (SEQ ID NO: 19) | (SEQ ID NO: 20) | (SEQ ID NO: 21) |
| M7H | ggctacatcttcaccaccta | attagtcctagcaacggtcg | gcacgattcgacgaggggga |
| | ctgg | ttct | cttc |
| | (SEQ ID NO: 22) | (SEQ ID NO: 23) | (SEQ ID NO: 24) |
| | GYIFTTYW | ISPSNGRS | ARFDEGDF |
| | (SEQ ID NO: 25) | (SEQ ID NO: 26) | (SEQ ID NO: 27) |
| M7Ha | GYIFITTW | ISPSNGRS | ARFDEGDF |
| | (SEQ ID NO: 64) | (SEQ ID NO: 26) | (SEQ ID NO: 27) |
| M1K1 | cagagtctgttaaacagtgg | ggggcatcc | cagaatgatcatagttatccatt |
| | aaatcaaaagacctac | (SEQ ID NO: 29) | cacg |
| | (SEQ ID NO: 28) | | (SEQ ID NO: 30) |
| | QSLLNSGNQKTY | GAS | QNDHSYPFT |
| | (SEQ ID NO: 31) | (SEQ ID NO: 32) | (SEQ ID NO: 33) |
| MIK1a | QSLLNSGNQKTY | GAS | QNDYSYPFT |
| | (SEQ ID NO: 31) | (SEQ ID NO: 32) | (SEQ ID NO: 65) |
| M1K2 | aaaagtgtcagtacatctgg | cttgtatcc | cagcacattagggagcttaca |
| | ctatagttat | (SEQ ID NO: 35) | cg |
| | (SEQ ID NO: 34) | | (SEQ ID NO: 36) |
| | KSVSTSGYSY | LVS | QHIRELT |
| | (SEQ ID NO: 37) | (SEQ ID NO: 38) | (SEQ ID NO: 39) |
| M2K | aaaagtgtcagtacatctgg | cttgtatcc | cagcacattagggagcttaca |
| | ctatagttat | (SEQ ID NO: 41) | cgt |
| | (SEQ ID NO: 40) | | (SEQ ID NO: 42) |
| | KSVSTSGYSY | LVS | QHIRELTR |
| | (SEQ ID NO: 43) | (SEQ ID NO: 44) | (SEQ ID NO: 45) |
| M1M7K | gagcctcttagaaagcgat | ctggtgtct | tggcaaggtacacattttccgt |
| | ggaaagacatat | (SEQ ID NO: 47) | ggacg |
| | (SEQ ID NO: 46) | | (SEQ ID NO: 48) |
| | QSLLESDGKTY | LVS | WQGTHFPWT |
| | (SEQ ID NO: 49) | (SEQ ID NO: 50) | (SEQ ID NO: 51) |

Cloned variable domains are shown in the charts below.

TABLE 4
DNA sequences of portions of the disclosed
anti-Cx43 antibodies. Variable heavy
chain (bold) and variable light chain (underlined).

| | | SEQ |
|---|---|---|
| mAb | Sequence | ID NO: |
| M1H | GAGGTCCAACTCCAGCAGCCTGGGGCTGAACTGGTG | 52 |
| | AAGCCTGGGGCTTCAGTGAAGTTGTCCTGCAAGGCTT | |
| | CTGGCTACACCTTCACCAGCTACTATATGTACTGGGT | |
| | GAAGCAGAGGCCTGGACAAGGCCTTGAGTGGATTGGG | |
| | GGAATTAATCCTAGCAATGGTGGTACTAACTTCAATG | |
| | AGAAGTTCAAGAACAAGGCCACACTGACTGTAGACAA | |
| | ATCCTCCAGCACAGCCTACATGCAACTCAGCAGCCTG | |
| | ACATCTGAGGACTCTGCGGTCTATTACTGTACAAGAG | |
| | AGGGTAACCCCTACTATACTATGAACTACTGGGGTCA | |
| | AGGAACCTCAGTCACCGTCTCCTCA | |
| M7H | GAGGTCCAACTCCAGCAACCTGGGGCTGAACTGGTG | 53 |
| | AGGCCTGGGGCTTCAGTAATGCTGTCCTGCAAGGCTT | |
| | CTGGCTACATCTTCACCACCTACTGGATGCACTGGCT | |
| | GAAGCAGAGGCCTGGACAAGGCCTTGACTGGATTGGA | |
| | GAGATTAGTCCTAGCAACGGTCGTTCTAATTACAATA | |
| | AGAAGTTCAAGAGCAAGGCCACACTGACTGTAGACAA | |
| | ATCCTCCAGCACAGCCTACATGCAACTCAGCAGCCTG | |
| | ACATCTGAGGACTCTGCGGTCTATTACTGTGCACGAT | |
| | TCGACGAGGGGGACTTCTGGGGCCAAGGCACCACTCT | |
| | CATAGTCTCCTCA | |
| M1K1 | GACATTGTGATGACGCAGTCTCCATCCTCCCTGAGT | 54 |
| | GTGTCAGCAGGAGAGAAGGTCACTATGAGCTGCAAGT | |
| | CCAGTCAGAGTCTGTTAAACAGTGGAAATCAAAAGAC | |
| | CTACTTGGCCTGGTACCAGCAGAAACCAGGGCAGCCT | |
| | CCTAAACTGTTGATCTACGGGGCATCCACTAGGGAAT | |
| | CTGGGGTCCCTGATCGCTTCACAGGCAGTGGATCTGG | |
| | AACCGATTTCACTCTTACCATCAGCAGTGTGCAGGCT | |
| | GAAGACCTGGCAGTTTATTACTGTCAGAATGATCATA | |
| | GTTATCCATTCACGTTCGGCTCGGGGACAAAGTTGGA | |
| | AATAAAA | |
| MIK2 | GACATTGTGTTGACACAGTCTCCTGCTTCCTTAGCTG | 55 |
| | TATCTCTGGGGCAGAGGGCCACCATCTCATACAGGGC | |
| | CAGCAAAAGTGTCAGTACATCTGGCTATAGTTATATG | |
| | CACTGGAACCAACAGAAACCAGGACAGCCACCCAGAC | |
| | TCCTCATCTATCTTGTATCCAACCTAGAATCTGGGGT | |
| | CCCTGCCAGGTTCAGTGGCAGTGGGTCTGGGACAGAC | |
| | TTCACCCTCAACATCCATCCTGTGGAGGAGGAGGATG | |
| | CTGCAACCTATTACTGTCAGCACATTAGGGAGCTTAC | |
| | ACGTTCGGAGGGGGGACCAAGCTGGAAATCAAAC | |
| M2K | GATATTGTGATGACCCAGTCTCCCGCTTCCTTAGCTG | 56 |
| | TATCTCTGGGGCAGAGGGCCACCATCTCATACAGGGC | |
| | CAGCAAAAGTGTCAGTACATCTGGCTATAGTTATATG | |
| | CACTGGAACCAACAGAAACCAGGACAGCCACCCAGAC | |
| | TCCTCATCTATCTTGTATCCAACCTAGAATCTGGGGT | |
| | CCCTGCCAGGTTCAGTGGCAGTGGGTCTGGGACAGAC | |
| | TTCACCCTCAACATCCATCCTGTGGAGGAGGAGGATG | |
| | CTGCAACCTATTACTGTCAGCACATTAGGGAGCTTAC | |
| | ACGTTCGGAGGGGGGGACCAAGCTGGAAATCAAA | |
| M1M7K | GACGTTGTGATGACCCAGACTCCACTCACTTTGTCGG | 57 |
| | TTACCATTGGACAACCAGCCTCCATCTCTTGCAAGTC | |
| | AAGTCAGAGCCTCTTAGAAAGCGATGGAAAGACATAT | |
| | TTGAATTGGTTGTTACAGAGGCCAGGCCAGTCTCCAA | |
| | AGCGCCTAATCTATCTGGTGTCTAAACTGGACTCTGG | |
| | AGTCCCTGACAGGTTCACTGGCAGTGGATCAGGGACA | |
| | GATTTCACACTGAAAATCAGCAGAGTGGAGGCTGAGG | |
| | ATTTGGGAGTTTATTATTGCTGGCAAGGTACACATTT | |
| | TCCGTGGACGTTCGGTGGAGGCACCAAGCTGGAAATC | |
| | AAA | |

Additional DNA sequences of portions of anti-Cx43 antibodies can be one or more the DNA sequences of portions of the disclosed anti-Cx43 antibodies disclosed in PCT/US2017/019605 (WO 2017/147561) which is hereby incorporated by references for its teaching of DNA sequences of portions of anti-Cx43 antibodies.

TABLE 5
Amino acid sequences of portions of the
disclosed anti-Cx43 antibodies.
Variable heavy chain (bold) and
variable light chain (underlined).

| | | SEQ ID |
|---|---|---|
| mAb | Sequence | NO: |
| MIH | EVQLQQPGAELVKPGASVKLSCKASGYTFTSYYMYWVKQR | 58 |
| | PGQGLEWIGGINPSNGGTNFNEKFKNKATLTVDKSSSTAY | |
| | MQLSSLTSEDSAVYYCTREGNPYYTMNYWGQGTSVTVSS | |
| M7H | EVQLQQPGAELVRPGASVMLSCKASGYIFTTYWMHWLKQR | 59 |
| | PGQGLDWIGEISPSNGRSNYNKKFKSKATLTVDKSSSTAY | |
| | MQLSSLTSEDSAVYYCARFDEGDFWGQGTTLIVSS | |
| MIK1 | DIVMTQSPSSLSVSAGEKVTMSCKSSQSLLNSGNQKTYLAW | 60 |
| | YQQKPGQPPKLLIYGASTRESGVPDRFTGSGSGTDFTLTIS | |
| | SVQAEDLAVYYCQNDHSYPFTFGSGTKLEIK | |
| M1K2 | DIVLTQSPASLAVSLGQRATISYRASKSVSTSGYSYMHWNQ | 61 |
| | QKPGQPPRLLIYLVSNLESGVPARFSGSGSGTDFTLNIHPV | |
| | EEEDAATYYCQHIRELTRSEGGPSWKSN | |
| M2-K | DIVMTQSPASLAVSLGQRATISYRASKSVSTSGYSYMHWNQ | 62 |
| | QKPGQPPRLLIYLVSNLESGVPARFSGSGSGTDFTLNIHPV | |
| | EEEDAATYYCQHIRELTRSEGGTKLEIK | |
| M1M7-K | DVVMTQTPLTLSVTIGQPASISCKSSQSLLESDGKTYLNWL | 63 |
| | LQRPGQSPKRLIYLVSKLDSGVPDRFTGSGSGTDFTLKISR | |
| | VEAEDLGVYYCWQGTHFPWTFGGGTKLEIK | |
| MIK1a | DIVMTQSPSSLSVSAGEKVTMSCKSSQSLLNSGNQKTYLAW | 66 |
| | YQQKPGQPPKLLIYGASTRESGVPDRFTGSGSGTDFTLTIS | |
| | SVQAEDLAVYYCQNDYSYPFTFGSGTKLEIK | |

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Treating Glaucoma, Optical Nerve Damage and Degeneration by Blocking Connexin 43 Hemichannels

Methods. Animals. WT and Cx43+/− mice were obtained by crossing WT mice with Cx43+/− mice (Reaume, A. G. et al. Science 267, 1831-1834 (1995)). Tail snip was used for obtaining DNA used for PCR analysis to confirm the genotype of WT and Cx43+/− mice. Mice were anesthetized with ketamine/xylazine or isoflurane and then eyes were dilated by applying one drop of 2.5% phenylephrine and one drop of 0.5% tropicamide.

Optic nerve crush. Optic nerve crush (ONC) was used to make optical nerve damage, retinal degeneration and ganglion cell death (Tang, Z. et al. J Vis Exp, doi: 10.3791/2685 (2011); and Cameron, E. G. et al. Bio Protoc 10, doi: 10.21769/BioProtoc.3559 (2020)). Briefly, mice at 3-month-old were anesthetized with 2.5% isoflurane, and then taped on a station under a dissection microscope with the mouse nose pointing toward the operator's dominant hand. The depth of anesthesia was observed before initiation and during surgical procedures. A small incision was made with iris scissors at the beginning of the conjunctiva inferior to the globe and around the eye temporally with caution to not cut too deep and risk damage to the underlying musculature and the supplying vasculature. To expose the posterior aspect of the globe and visualize the optic nerve, the globe was retracted and rotated nasally with a micro-forceps to grasp the edge of the conjunctiva. A Dumont #N7 cross-action forceps (cat. #RS-5027; Roboz) was inserted to grasp the exposed optic nerve at approximately 1-3 mm from the globe for 3 second with pressure being from the self-clamping action of the forceps to press on the nerve. The Dumont cross-action forceps was used because its spring action applied a constant and consistent force to the optic nerve. After 3 seconds, the optic nerve was released and the forceps were removed, allowing the eye to rotate back into place. At the end of the procedure, a drop of 0.5% proparacaine hydrochloride ophthalmic solution was administered for post-operative pain control, and a small amount of surgical lubricant was applied to the eye to protect it from drying. The mice were placed on a warming pad and monitored until it fully recovered from anesthesia. Then mice were transferred into a regular cage and monitored for post-operative complications every day until the end of experiment.

The M1 antibody (25 μg/ml of vitreous body or saline) in 1 μl was injected by intravitreous injection 30 minutes after ONC.

SO-induced ocular hypertension under-detected (SOHU). This model can consistently increase intraocular pressure, mimicking human glaucoma (Fang, F. et al. Scientific reports 11, 9052 (2021); Zhang, J. et al., eLife 8 (2019); and Zhang, J. et al. J Vis Exp, (2019)). SOHU is a simple and stable intraocular pressure (IOP) model that increases IOP mainly through anterior chamber injection of silicone oil (˜2 μL, the sizes of SO droplet >1.5 mm). SO can cause an effective pupillary obstruction. When SO is injected into the anterior chamber, it forms a droplet that comes in contact with the iris surface and tightly seals the pupil due to high surface tension, resulting in disruption of atrial aqueous circulation and causing an increase in IOP. The ciliary body continuously produces aqueous humor that accumulates in the posterior chamber, pushing the iris forward. When the iris root contacts the posterior surface of the cornea, the anterior chamber angle closes. The closure of the atrial angle further impedes the outflow of aqueous fluid through the trabecular meshwork (TM) and may also lead to an increase in IOP. IOP elevation in the SO eye started as early as 2 days post injection (2 dpi) and remained elevated for at least 8 weeks. Ultimately, this model causes visual function defects in mice, retinal functional impairment, and glaucomatous degeneration of retinal ganglion cell somata and axons.

The M1 antibody (25 μg/ml of vitreous body or saline) in 1 μl were injected by intravitreous injection 2 days post SO injection.

Intravitreous injection. Mice were anesthetized by intra-peritoneal injection of ketamine (100 mg/kg) and xylazine (20 mg/kg). The topical anesthetization was used by applying one drop of 0.5% alcaine to the mouse eyes before intravitreous injection. The depth of anesthesia was monitored before initiation and during surgical procedures. Injection into the vitreous cavity was performed using a 33-gauge needle and a micropipette on the ONC eye. Injection was performed under an ophthalmic operating microscope with a micropipette at a location approximately 1 mm behind limbus. M1 antibody (25 mg/kg) or saline with a volume at 1 μl was injected into the vitreous cavity. The needle was kept in place for 10 seconds and then gently removed out. After intravitreous injection, ophthalmic ointment was applied on the mouse eye to prevent infection.

Optical Coherence Tomography. Mice were imaged at different times (before and 4 weeks after ONC). Mice were anesthetized with intraperitoneal ketamine (100 mg/kg) and xylazine (20 mg/kg) and their pupils were dilated. The anesthetized mice were set on the stage of the OCT imaging system (Micron®III Phoenix In) and their retinal structures were assessed using the Micron. The scanning pattern is horizontal. For each retina, at least 3 measurements were taken in the cross-section images with definable boundary using the Phoenix InSight software.

Electroretinogram. electroretinogram (ERG) measurements were conducted (Liu, Y. et al. Invest Ophthalmol Vis Sci 55, 3766-3774 (2014)). Briefly, full field flash ERG was performed with the Handheld Multispecies-ElectroRetinoGraph (HMsERG) unit (Ocuscience, Henderson, NV, USA) using polymethylmethacrylate (acrylic) and gold electrodes (LKC, Gaithersburg, USA). Mice were dark adapted overnight before the ERG recording. Isoflurane inhalation anesthesia was maintained during the ERG recording period. The procedures were performed in a dark room with a dim red safe light as needed. Body temperature was maintained by a heating pad. Pupils were dilated with 1% tropicamide ophthalmic solution. The electrodes were adhered and covered the entire cornea. The cornea was kept moist with 0.9% sterile saline. Two white needle reference electrodes were subdermally inserted into each cheek above the jaw line in an anterior direction. Another needle electrode, which served as ground electrode, was inserted into the skin at the tail base. Bilateral ERG recording was performed from both eyes simultaneously. The light intensity for the positive scotopic threshold response (pSTR) test was 3×10⁻⁵ cd·s·m⁻², and responses from 10 flashes with 2-second intervals were averaged. The mice were subjected to the light adaptation for 15 min after the scotopic ERG measurement, and the background light intensity was 30 cd·s·m⁻². The light intensity for the photopic negative response (PhNR) test was 15 cd·s·m⁻². Responses from 32 flashes with 0.75-second intervals were averaged. The pSTRs were measured from the baseline to the positive peak of the waveform and the PhNRs were measured from the baseline to the trough of the negative response following the positive b-wave.

Retinal wholemount preparation and staining. Eyes were enucleated and the cornea, lens and vitreous humor were removed. The dorsal part of the retina was notched to retain orientation. The retina and sclera were fixed in 4% paraformaldehyde in PBS for 30 minutes at room temperature. Four cuts were made around the circumference of the retina creating four quadrants to flatten the retina on a slide when mounted. The retina was then carefully removed from the sclera. To detect SMI-32 expression (Akopian, A. et al. J Clin Invest 127, 2647-2661 (2017)), retinal were blocked in PBS containing 10% normal donkey serum (NDS), 1% BSA, and 0.5% Triton X-100 for 1 hr. The tissues were then incubated with anti-SMI-32 antibody (1:200, 801701, BioLegend, Inc. CA, USA) diluted in containing 3% NDS, 1% BSA, and 0.1% Triton X-100 for 48 hours at 4° C. To detect Brn-3a expression (Kerr, N. M. et al. Exp Neurol 234, 144-152, (2012)), the retina was permeabilized with 0.5% Triton X-100 in PBS for 15 min at −80° C. and then incubated in 2% Triton-X100 in PBS overnight at 4° C. Following a thorough wash with PBS, free floating retinas were incubated overnight at 4° C. in PBS containing mouse anti-BRN3a primary antibody (1:100 dilution) (sc-8429, Santa-Cruz Biotechnology, Dallas, Texas, U.S.A), 2% donkey serum and 2% Triton-X100. After additional wash with PBS, the retinas were incubated for 3 hours at room temperature in PBS solution containing 2% NDS, donkey anti-mouse secondary antibody conjugated to Alexa Fluor 488 (1:400 dilution) to detect BRN3a or Alexa Fluor 594 (1:400 dilution) to detect SMI-32, respectively. Retinas were mounted onto SuperFrost Plus slides using OCT compound mounting medium (Sakura, Torrance, CA, USA). Images of immunolabeled tissues were taken by a Keyence BZ-X710 All-in-One fluorescence microscopy with 20× or 60× (oil immersion) objectives. High-resolution (1,024×1,024 pixels) Z-stack images were taken using step size of 0.7-2.0 μm, compiled to a single plane, and analyzed quantitatively.

Frozen section. Eye ball was fixed with 2% PFA for 4 hours at RT and then submersed into the sucrose gradient solutions of 10%, 20%, and 30% sucrose for 30 min, 1 hour, and overnight, respectively at 4° C. The eyeballs were transferred into the cryostat chamber and add OCT medium setting for 2 hrs at RT. The cryostat chamber was quickly frozen and kept in-80 freezer. The frozen sections were cut with 10 μm thickness by a cryostat (HM505) at −20° C. and mounted onto gelatin coated slides. The slides were kept at −20° C. until use. The slides were postfixed in −20° C. acetone for 5 minutes and air dried at RT for 5 minutes. An encircle around tissue was drawn with a liquid blocker to create a barrier for further incubation. The tissues were rinsed with PBS for 5 min for 3 times, blocked with block solution for 1 hour at RT, incubated in primary antibody for overnight, rinsed with PBS for 5 min for 3 times, incubated in Alex 488 or 549 conjugated secondary antibodies for 1 hour at RT, rinsed with PBS for 5 min for 3 times and sealed coverslip with mounting medium. Images were taken under Keyence microscope (BZ Viewer, KEYENCE CORPORATION OF AMERICA) and analyzed with Image J (NIH).

Western blot analysis. Retinal were washed with cold PBS and then lysed in RIPA buffer (50 mM Tris, pH 7.5, 150 mM NaCl, 1 mM EDTA, 1% Triton X-100, 0.5% NaDOC, and 0.1% SDS) supplemented with protease and phosphatase inhibitors. Retinal homogenates were incubated on ice for 30 min and then centrifuged at 16,600 g for 10 min. The resulting supernatants were collected and assayed for protein concentration using BCA protein assay reagent (Pierce, Rockford, IL, USA). To examine the expression level of GFAP and Cx43, 20-30 μg of protein was loaded on SDS-PAGE and transferred to PVDF membranes. The membranes were blocked with 5% skim milk and treated with chicken anti-GFAP (1:1,000 AB5541 Millipore Sigma, Burlington, USA), rabbit anti-Cx43 (1:1,000) and mouse anti-GAPDH (1:5000) antibodies (HyTest, Waltham, USA). The secondary antibodies were donkey anti-rabbit and anti-chicken horseradish-peroxidase conjugated (Sigma-Aldrich). The intensity of the bands was quantified using an Image Studio Lite Ver 5.2 software.

Preparation of primary astrocyte cell culture. The brain tissue was isolated from <3 days old mouse, rinsed with chilled and was titrated up and down with pipet astrocyte medium (AM, Innoprot) until no tissue chunks were visible. The cell suspension was filtered through a mesh strainer and cultured in one tissue culture flask at 5% CO₂ incubator at 37° C. overnight. The next day, the medium was collected and placed on a new culture flask with addition of fresh 5-10 ml astrocyte medium and incubated for overnight. This was the secondary plating. The previous flask was also treated to remove any leftover neurons by incubating with fresh astrocyte medium containing serum FBS/FCS and treated with cold temperature (room temperature) to remove additional neuronal cells. The culture continued for 10 days and half amount of media was changed every 3-4 days. Through this panning method, neuronal cells were removed and primary astrocyte cell culture was established.

Differentiation of R28 cells into retinal ganglion cell (RGC)-like cells. R28 Cells were cultured in low glucose DMEM with 10% FBS, 1% L-glutamine (Sigma), 1% non-essential amino acids (Sigma), and 50 μg gentamicin. R28 Cells were seeded at 2×10⁴ cells/cm² on a 60-mm dish coated with laminin and induced with 250 M pCPT-cyclic AMP (a cell-permeable cAMP analogue) for 24 hours.

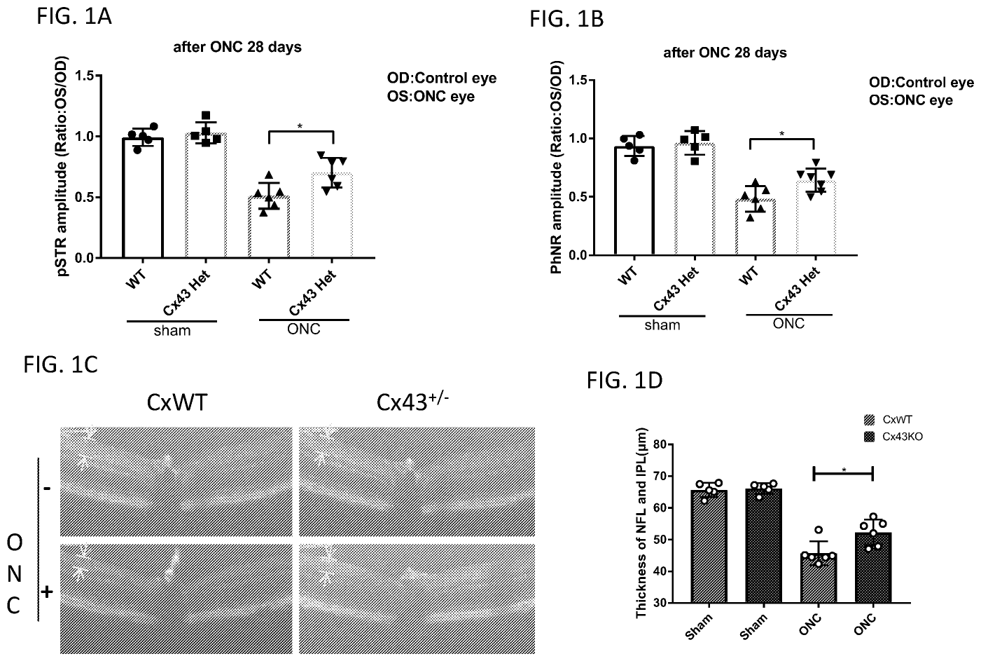

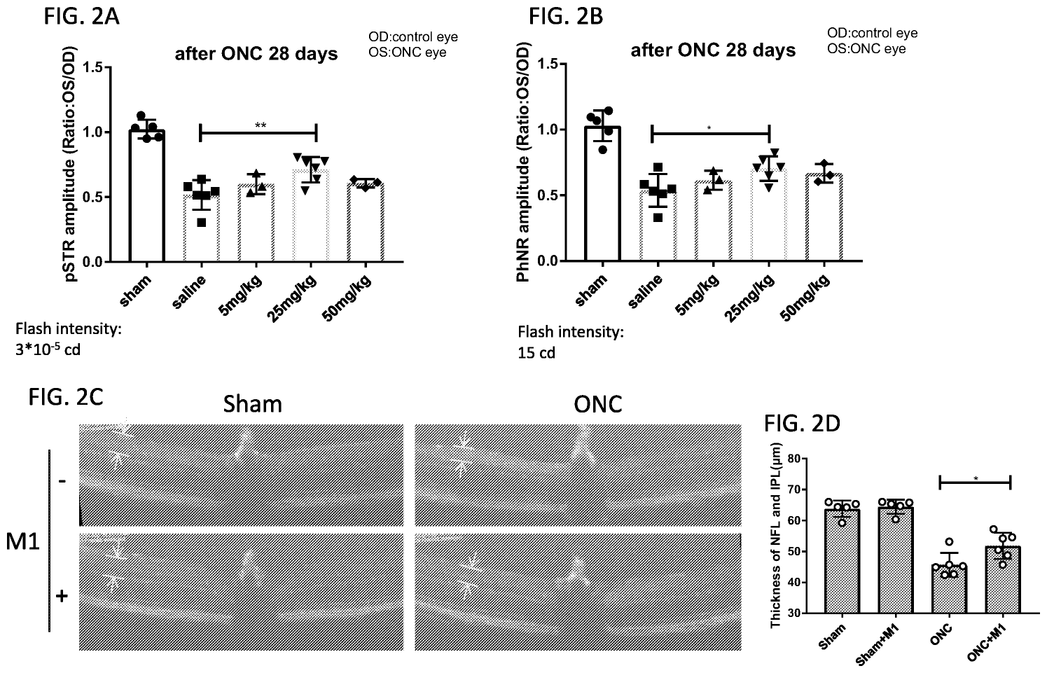

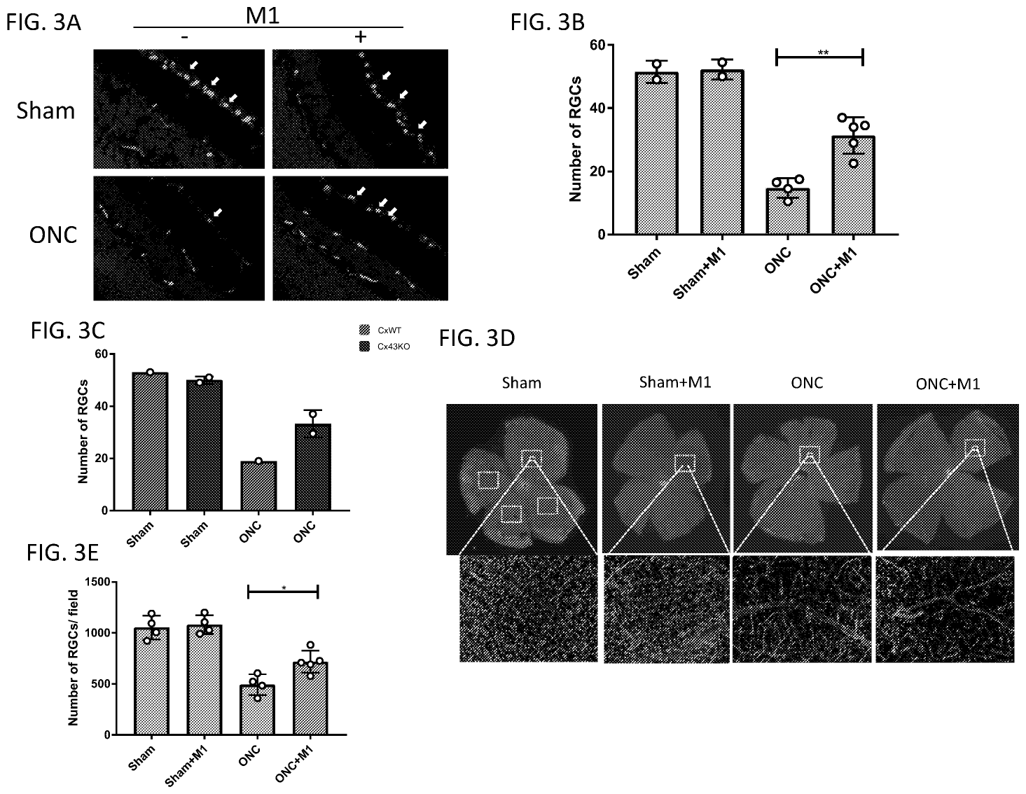

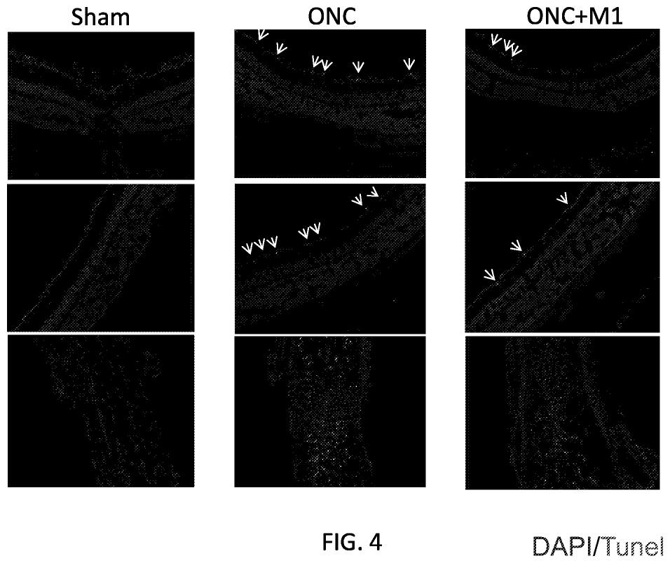

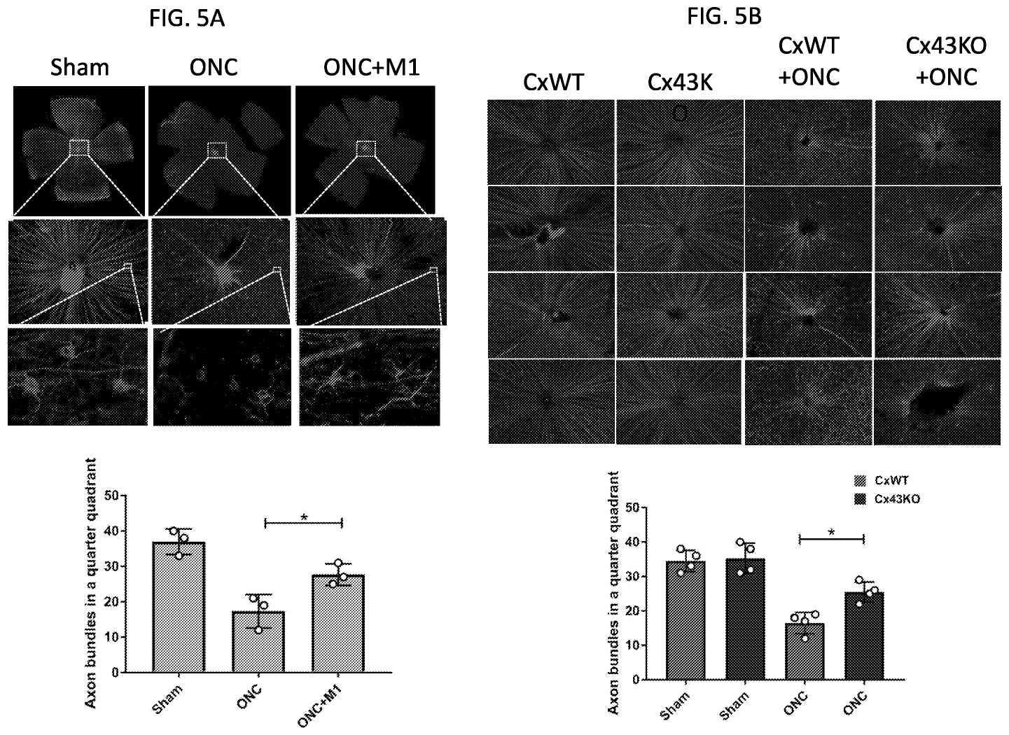

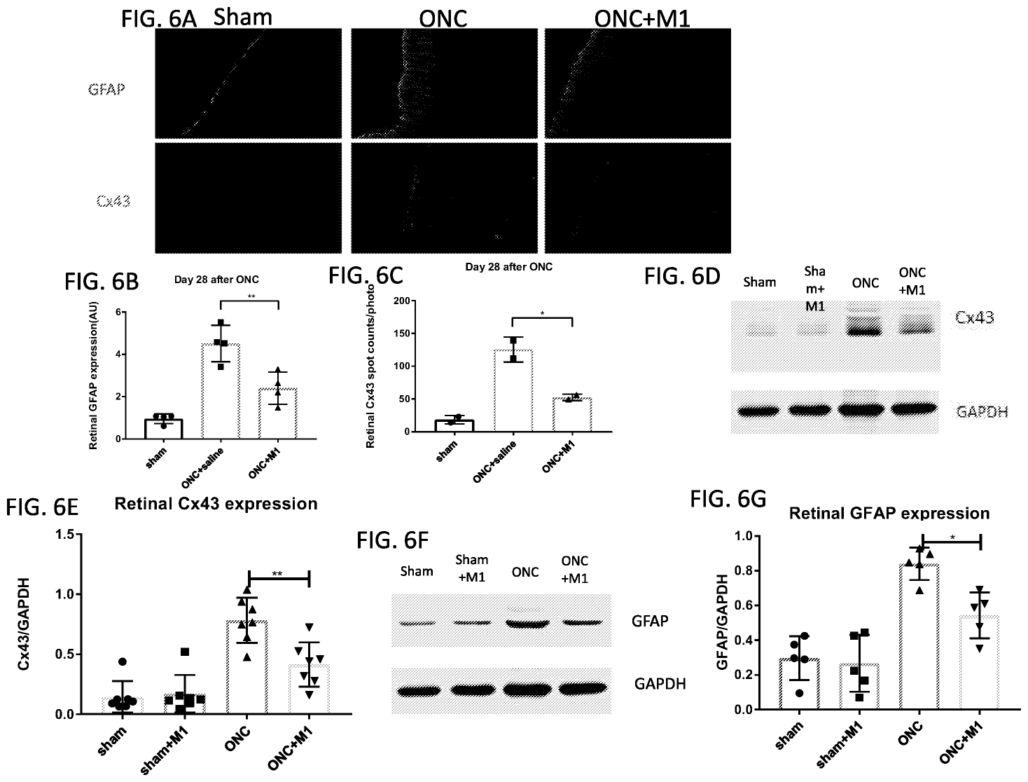

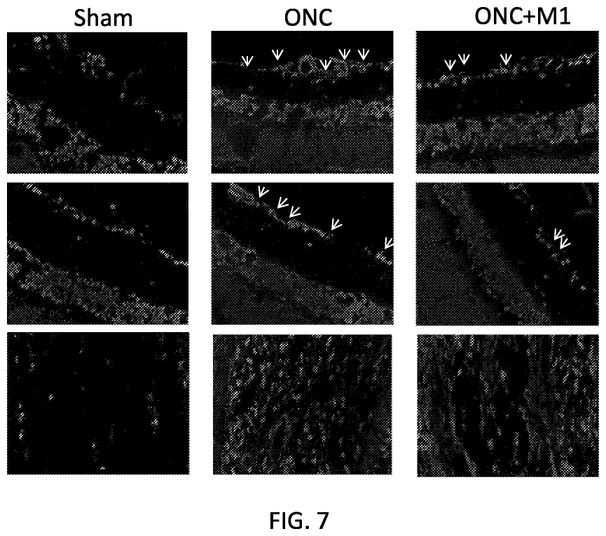

Results. FIGS. 1A-D show haplosufficiency of Cx43 reduces retinal function loss and retinal injury after optical never crush (ONC). FIGS. 2A-D show inhibition of Cx43 hemichannels by M1 antibody protects visual function after ONC. FIGS. 3A-D show that M1 antibody reduces retinal ganglion cell loss. FIG. 4 shows that the M1 antibody reduces apoptosis after ONC in the retina. FIGS. 5A-B show that the M1 antibody preserves RGC axon, somata and dendrites after ONC in the retina. FIGS. 6A-G show that the M1 antibody reduces reactive gliosis after ONC. FIG. 7 shows that M1 antibody reduces microglia and neuroinflammation after ONC in retinal sections.

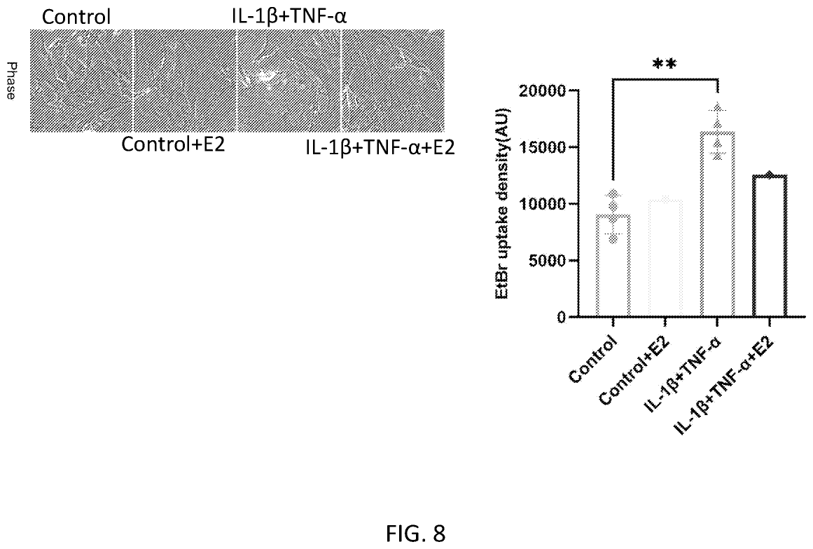

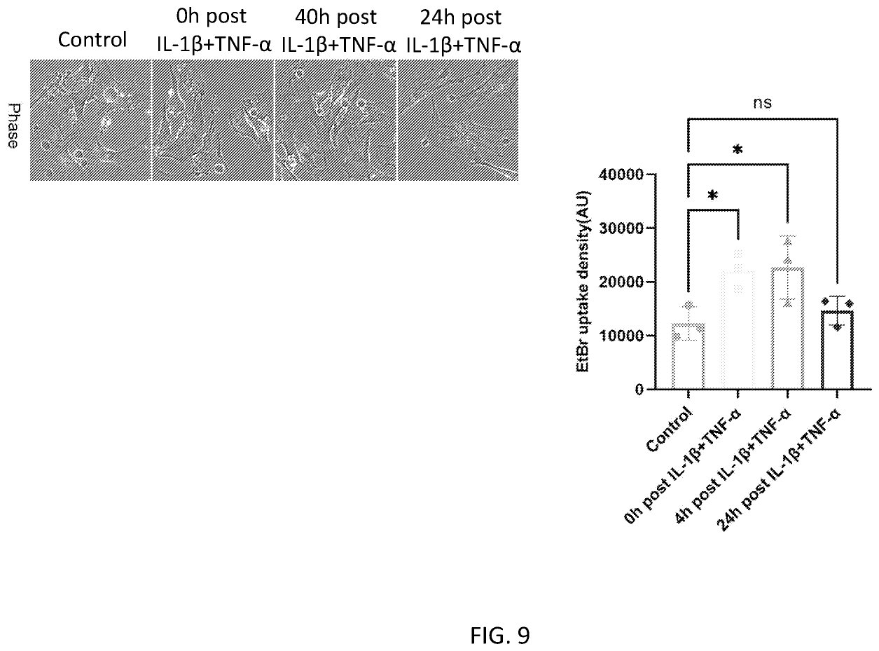

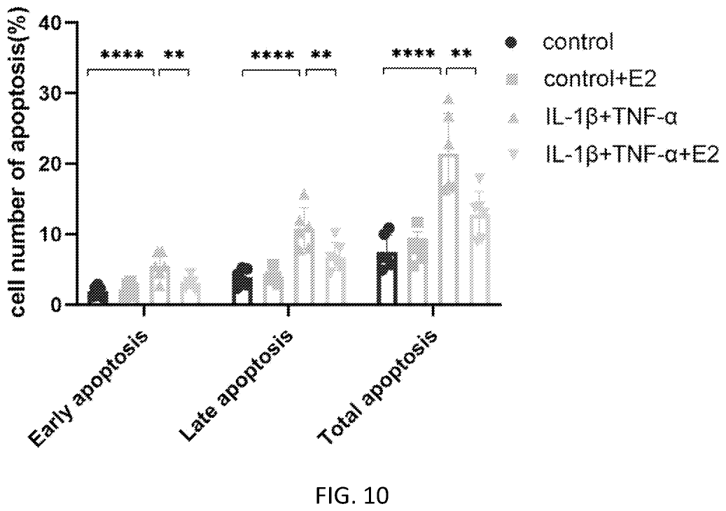

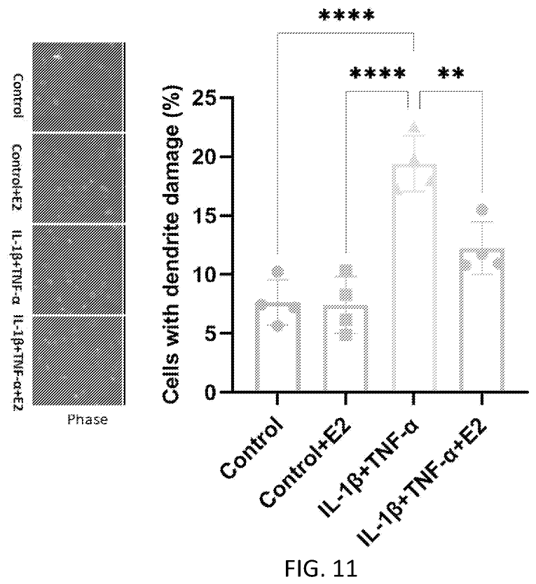

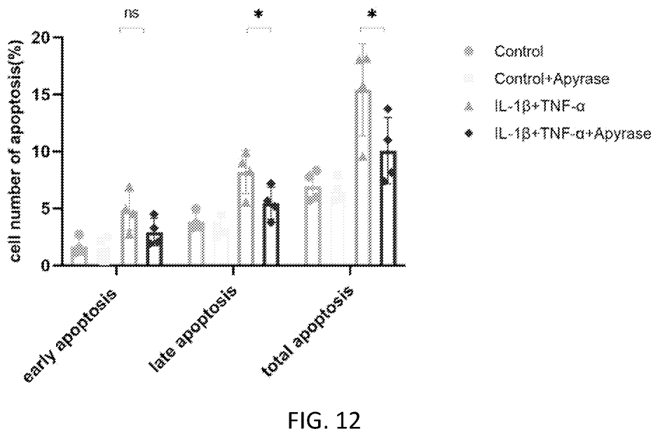

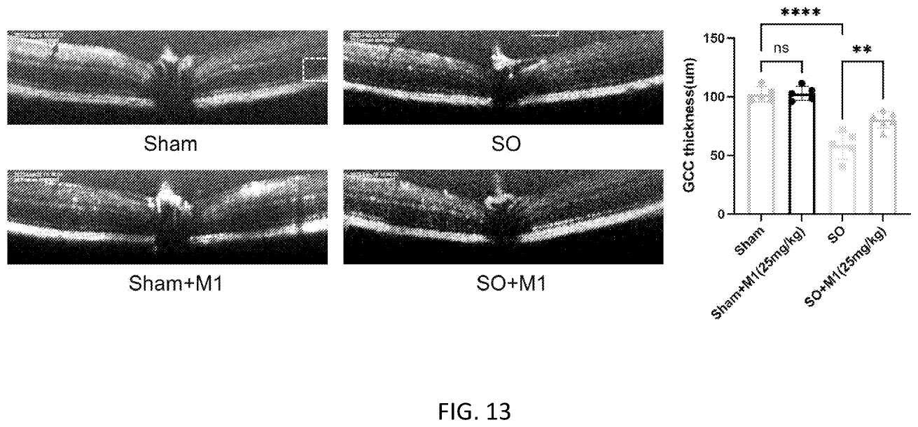

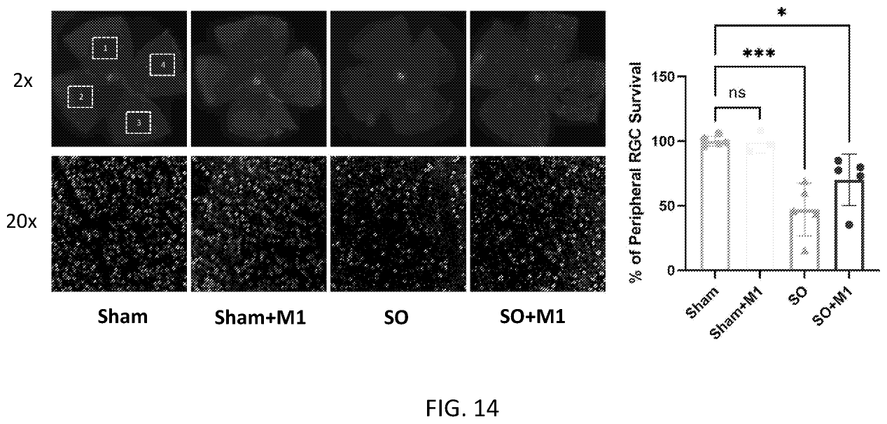

FIG. 8 shows that Cx43 hemichannel opening in astrocytes induced by inflammatory factor, interleukin-1 beta/TNG-alpha is inhibited by an anti-Cx43 hemichannel blocking antibody (E2). E2 is a polyclonal antibody against the E2 extracellular loop that binds to Cx43 hemichannels. FIG. 9 shows stained opening of astrocyte Cx43 in response to cytokines IL-1 beta/TNF-alpha treatment. Even after removal of IL-1 beta/TNF-alpha, hemichannel-mediate dye uptake was shown 4 hour post treatment. FIG. 10 shows inhibition of astrocyte Cx43 hemichannels protects retinal ganglion cells (RGCs). FIG. 11 shows inhibition of Cx43 hemichannels in astrocytes protect RGC dendritic damages caused by inflammatory cytokines IL-1 beta/TNF-alpha. FIG. 12 shows that Cx43 hemichannel releases ATP and extracellular ATP increases RGC-like R28 cell apoptosis. FIG. 13 shows inhibition of Cx43 hemichannels by a monoclonal anti-Cx43 hemichannel blocking antibody (the M1 antibody) protects RGC degradation induced by increased intraocular pressure (IOP) in silicon oil (SO) injection mouse model. FIG. 14 shows that inhibition of Cx43 hemichannels by an anti-Cx43 hemichannel antibody (the M1 antibody) protects RGC degeneration induced by elevated IOP in silicon oil (SO) injection mouse model.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. A method of treating or preventing glaucoma in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

2. A method of treating or preventing macular degeneration in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

3. A method of treating or preventing an eye disease or an eye injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

4. A method of treating or preventing diabetic retinopathy in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

5. A method of treating or preventing retinal injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

6. A method of treating or preventing optical nerve damage or degeneration in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

7. A method of treating or preventing glaucoma in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

8. A method of treating or preventing macular degeneration in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

9. A method of treating or preventing an eye disease or an eye injury in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

10. A method of treating or preventing diabetic retinopathy in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

11. A method of treating or preventing retinal injury in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

12. A method of treating or preventing optical nerve damage or degeneration in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

13. A method of treating or preventing glaucoma in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

14. A method of treating or preventing macular degeneration in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

15. A method of treating or preventing an eye disease or an eye injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

16. A method of treating or preventing diabetic retinopathy in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

17. A method of treating or preventing retinal injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

18. A method of treating or preventing optical nerve damage or degeneration in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

19. A method of reducing retinal ganglion cell loss or reducing retinal ganglion apoptosis, the method comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

20. A method of protecting or preserving retinal ganglion cell axon, somata or dendrites in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

21. A method of reducing reactive gliosis in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

22. A method of reducing microglia activation or neuroinflammation in the retina in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

23. A method of reducing optical nerve damage and degeneration in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

24. A method of protecting visual function in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

25. A method of reducing retinal ganglion cell loss or reducing retinal ganglion apoptosis, the method comprising administering to a subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

26. A method of protecting or preserving retinal ganglion cell axon, somata or dendrites in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

27. A method of reducing reactive gliosis in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

28. A method of reducing microglia activation or neuroinflammation in the retina in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

29. A method of reducing optical nerve damage and degeneration in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

30. A method of protecting visual function in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

31. A method of reducing retinal ganglion cell loss or reducing retinal ganglion apoptosis, the method comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

32. A method of protecting or preserving retinal ganglion cell axon, somata or dendrites in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

33. A method of reducing reactive gliosis in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

34. A method of reducing microglia activation or neuroinflammation in the retina in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

35. A method of reducing optical nerve damage and degeneration in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

36. A method of protecting visual function in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

37. A method of reducing or ameliorating one or more symptoms of an eye disease or an eye injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

38. A method of reducing or ameliorating one or more symptoms of an eye disease or an eye injury in a subject, the method comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

39. A method of reducing or ameliorating one or more symptoms of an eye disease or an eye injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

40. The method of any one of the preceding claims, wherein the antibody or fragment thereof comprises a heavy chain immunoglobulin variable region comprising:
a) a complementarity determining region 1 (CDR1) comprising the sequence of SEQ ID NO: 19 or a variant thereof;
b) a complementarity determining region 2 (CDR2) comprising the sequence of SEQ ID NO: 20 or a variant thereof; and/or
c) a complementarity determining region (CDR3) comprising the sequence of SEQ ID NO: 21 or a variant thereof.

41. The method of any one of the preceding claims, wherein the antibody or fragment thereof comprises a light chain immunoglobulin variable region comprising:
a) a complementarity determining region 1 (CDR1) comprising the sequence of SEQ ID NO: 31 or a variant thereof;
b) a complementarity determining region 2 (CDR2) comprising the sequence of SEQ ID NO: 32 or a variant thereof; and/or
c) a complementarity determining region 3 (CDR3) comprising the sequence of SEQ ID NO: 33 a variant thereof.

42. The method of claim 40 or 41, wherein any one of the CDR1, CDR2 or CDR3 of the heavy chain immunoglobulin variable region or the CDR1, CDR2, or CDR3 of the light chain immunoglobulin variable region comprise at least one amino acid substitution as compared to the parent CDR.

43. The method of claim 42, wherein the at least one amino acid substitution is a cysteine residue to another amino acid or a glycine to another amino acid.

44. The method of any of the preceding claims, wherein the antibody or fragment thereof comprises a heavy chain immunoglobulin variable region comprising:
(a) a complementarity determining region 1 (CDR1) comprising a sequence having at least 60% identity to a sequence set forth in SEQ ID NO: 19;
(b) a complementarity determining region 2 (CDR2) comprising a sequence having at least 60% identity to a sequence set forth in SEQ ID NO: 20; and/or
(c) a complementarity determining region 3 (CDR3) comprising a sequence having at least 60% identity to a sequence set forth in SEQ ID NO: 21.

45. The method of any of the preceding claims, wherein the antibody or fragment thereof comprises a light chain immunoglobulin variable region comprising:
(a) a complementarity determining region 1 (CDR1) comprising a sequence having at least 60% identity to a sequence set forth in SEQ ID NO: 31;
(b) a complementarity determining region 2 (CDR2) comprising a sequence having at least 60% identity to a sequence set forth in SEQ ID NO: 32; and/or
(c) a complementarity determining region 3 (CDR3) comprising a sequence having at least 60% identity to a sequence set forth in SEQ ID NO: 33.

46. The method of any of the claims 1-45, wherein the therapeutically effective amount of the antibody or the fragment thereof is in an expression vector encoding the antibody or the fragment thereof.

47. The method of any of claims 1-45, wherein the antibody is administered in a pharmaceutically acceptable composition.

48. The method of any of claim 1-45 or 47, wherein the antibody is administered locally.

49. The method of any of claim 1-45 or 47, wherein the antibody is administered via intraocular administration.

50. The method of any of the preceding claims, wherein the antibody is a humanized antibody.

51. The method of any of the preceding claims, further comprising administering at least a second therapeutic agent to the subject.

52. The method of any of any of the preceding claims, wherein the antibody or fragment thereof binds to a Cx43 hemichannel.

53. The method of any of any of the preceding claims, wherein the antibody or fragment thereof further comprises a tag sequence.

54. The method of any of any of the preceding claims, wherein the antibody or fragment thereof inhibits or blocks the opening of a Cx43 hemichannel.

55. The method of any of any of the preceding claims, wherein the fragment thereof is a Fab fragment, an Fab′ fragment or an F(ab′)2 fragment.

56. The method of claim 47, wherein the pharmaceutical composition is lyophilized.

57. The method of any of the preceding claims, wherein the antibody or fragment thereof comprises:
(a) a heavy chain immunoglobulin variable region comprising:
i) a first complementarity determining region 1 comprising a sequence having at least 60% identity to SEQ ID NO: 19;
ii) a second complementarity determining region 2 comprising a sequence having at least 60% identity to SEQ ID NO: 20; and
iii) a third complementarity determining region 3 comprising a sequence having at least 60% identity to SEQ ID NO: 21; and
(b) a light chain immunoglobulin variable region comprising:
i) a first complementarity determining region 1 comprising a sequence having at least 60% identity to SEQ ID NO: 31;
ii) a second complementarity determining region 1 comprising a sequence having at least 60% identity to SEQ ID NO: 32; and
iii) a third complementarity determining region 1 comprising a sequence having at least 60% identity to SEQ ID NO: 33.

58. The method of claim 57, wherein the antibody is a humanized antibody.

59. The method any of claims 37-39, wherein the one or more symptoms as of the eye disease or eye injury is vision loss.

60. The method of any of the preceding claims, wherein the antibody or fragment thereof comprises a heavy chain immunoglobulin variable region comprising:
(a) a complementarity determining region 1 (CDR1) comprising a sequence having a single amino acid change compared to a sequence set forth in SEQ ID NO: 19;
(b) a complementarity determining region 2 (CDR2) comprising a sequence having a single amino acid change compared to a sequence set forth in SEQ ID NO: 20; and/or
(c) a complementarity determining region 3 (CDR3) comprising a sequence having a single amino acid change compared to a sequence set forth in SEQ ID NO: 21.

61. The method of any of the preceding claims, wherein the antibody or fragment thereof comprises a light chain immunoglobulin variable region comprising:
(a) a complementarity determining region 1 (CDR1) comprising a sequence having a single amino acid change compared to a sequence set forth in SEQ ID NO: 31;
(b) a complementarity determining region 2 (CDR2) comprising a sequence having a single amino acid change compared to a sequence set forth in SEQ ID NO: 32; and/or
(c) a complementarity determining region 3 (CDR3) comprising a sequence having a single amino acid change compared to a sequence set forth in SEQ ID NO: 33.

62. The method of any of the preceding claims, wherein the antibody or fragment thereof comprises:
(a) a heavy chain immunoglobulin variable region comprising:
i) a first complementarity region 1 comprising a sequence having a single amino acid change compared to SEQ ID NO: 19;
ii) a second complementarity region 2 comprising a sequence having a single amino acid change compared to SEQ ID NO: 20; and
iii) a third complementarity region 3 comprising a sequence having a single amino acid change compared to SEQ ID NO: 21; and
(b) a light chain immunoglobulin variable region comprising:
i) a first complementarity region 1 comprising a sequence having a single amino acid change compared to SEQ ID NO: 31;
ii) a second complementarity region 1 comprising a sequence having a single amino acid change compared to SEQ ID NO: 32; and
iii) a third complementarity region 1 comprising a sequence having a single amino acid change compared to SEQ ID NO: 33.


SEQUENCE LISTING (79 sequences)

SEQ ID NO: 1 - DNA, 384 bp, Mus musculus
  source 1..384: mol_type = other DNA
gaggttcagctggagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctcagccaaaacgacacccccatctgtctat

SEQ ID NO: 2 - protein, 128 aa, synthetic construct
EVQLEQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSSAKTTPPSVY

SEQ ID NO: 3 - DNA, 324 bp, synthetic construct
  source 1..324: mol_type = other DNA
gatattgtgatgacacagactcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaa

SEQ ID NO: 4 - protein, 108 aa, synthetic construct
DIVMTQTPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 5 - DNA, 384 bp, synthetic construct; identical to SEQ ID NO: 1
  source 1..384: mol_type = other DNA

SEQ ID NO: 6 - protein, 128 aa, synthetic construct; identical to SEQ ID NO: 2

SEQ ID NO: 7 - DNA, 375 bp, synthetic construct
  source 1..375: mol_type = other DNA
gatattgtgatgacccagactccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaacgggctgatgctgcaccaactgtatccgcatgcacc

SEQ ID NO: 8 - protein, 122 aa, synthetic construct
DIVMTQTPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKLEIKRADAAPTVS

SEQ ID NO: 9 - DNA, 405 bp, synthetic construct
  source 1..405: mol_type = other DNA
ctggagcagcctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctcagccaaaacaacagccccatcggtctatccactggcccctgtgtgtggagatacaactggctcctcggtg

SEQ ID NO: 10 - protein, 135 aa, synthetic construct
LEQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVSSAKTTAPSVYPLAPVCGDTTGSSV

SEQ ID NO: 11 - DNA, 324 bp, synthetic construct; identical to SEQ ID NO: 3
  source 1..324: mol_type = other DNA

SEQ ID NO: 12 - protein, 108 aa, synthetic construct; identical to SEQ ID NO: 4

SEQ ID NO: 13 - protein, 10 aa, synthetic construct
FLSRPTEKTI

SEQ ID NO: 14 - protein, 10 aa, synthetic construct
KRDPCPHQVD

SEQ ID NO: 15 - protein, 9 aa, synthetic construct
LSAVYTCKR

SEQ ID NO: 16 - DNA, 24 bp, synthetic construct
  source 1..24: mol_type = other DNA
ggctacaccttcaccagctactat

SEQ ID NO: 17 - DNA, 24 bp, synthetic construct
  source 1..24: mol_type = other DNA
attaatcctagcaatggtggtact

SEQ ID NO: 18 - DNA, 36 bp, synthetic construct
  source 1..36: mol_type = other DNA
acaagagagggtaacccctactatactatgaactac

SEQ ID NO: 19 - protein, 8 aa, synthetic construct
GYTFTSYY

SEQ ID NO: 20 - protein, 8 aa, synthetic construct
INPSNGGT

SEQ ID NO: 21 - protein, 12 aa, synthetic construct
TREGNPYYTMNY

SEQ ID NO: 22 - DNA, 24 bp, synthetic construct
  source 1..24: mol_type = other DNA
ggctacatcttcaccacctactgg

SEQ ID NO: 23 - DNA, 24 bp, synthetic construct
  source 1..24: mol_type = other DNA
attagtcctagcaacggtcgttct

SEQ ID NO: 24 - DNA, 24 bp, synthetic construct
  source 1..24: mol_type = other DNA
gcacgattcgacgagggggacttc

SEQ ID NO: 25 - protein, 8 aa, synthetic construct
GYIFTTYW

SEQ ID NO: 26 - protein, 8 aa, synthetic construct
ISPSNGRS

SEQ ID NO: 27 - protein, 8 aa, synthetic construct
ARFDEGDF

SEQ ID NO: 28 - DNA, 36 bp, synthetic construct
  source 1..36: mol_type = other DNA
cagagtctgttaaacagtggaaatcaaaagacctac

SEQ ID NO: 29 - skipped

SEQ ID NO: 30 - DNA, 27 bp, synthetic construct
  source 1..27: mol_type = other DNA
cagaatgatcatagttatccattcacg

SEQ ID NO: 31 - protein, 12 aa, synthetic construct
QSLLNSGNQKTY

SEQ ID NO: 32 - skipped

SEQ ID NO: 33 - protein, 9 aa, synthetic construct
QNDHSYPFT

SEQ ID NO: 34 - DNA, 30 bp, synthetic construct
  source 1..30: mol_type = other DNA
aaaagtgtcagtacatctggctatagttat

SEQ ID NO: 35 - skipped

SEQ ID NO: 36 - DNA, 23 bp, synthetic construct
  source 1..23: mol_type = other DNA
cagcacattagggagcttacacg

SEQ ID NO: 37 - protein, 10 aa, synthetic construct
KSVSTSGYSY

SEQ ID NO: 38 - skipped

SEQ ID NO: 39 - protein, 7 aa, synthetic construct
QHIRELT

SEQ ID NO: 40 - DNA, 30 bp, synthetic construct; identical to SEQ ID NO: 34
  source 1..30: mol_type = other DNA

SEQ ID NO: 41 - skipped

SEQ ID NO: 42 - DNA, 24 bp, synthetic construct
  source 1..24: mol_type = other DNA
cagcacattagggagcttacacgt

SEQ ID NO: 43 - protein, 10 aa, synthetic construct
KSVSTSGYSY

SEQ ID NO: 44 - skipped

SEQ ID NO: 45 - protein, 8 aa, synthetic construct
QHIRELTR

SEQ ID NO: 46 - DNA, 31 bp, synthetic construct
  source 1..31: mol_type = other DNA
gagcctcttagaaagcgatggaaagacatat

SEQ ID NO: 47 - skipped

SEQ ID NO: 48 - DNA, 27 bp, synthetic construct
  source 1..27: mol_type = other DNA
tggcaaggtacacattttccgtggacg

SEQ ID NO: 49 - protein, 11 aa, synthetic construct
QSLLESDGKTY

SEQ ID NO: 50 - skipped

SEQ ID NO: 51 - protein, 9 aa, synthetic construct
WQGTHFPWT

SEQ ID NO: 52 - DNA, 357 bp, synthetic construct
  source 1..357: mol_type = other DNA
gaggtccaactccagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctca

SEQ ID NO: 53 - DNA, 345 bp, synthetic construct
  source 1..345: mol_type = other DNA
gaggtccaactccagcaacctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctca

SEQ ID NO: 54 - DNA, 339 bp, synthetic construct
  source 1..339: mol_type = other DNA
gacattgtgatgacgcagtctccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaa

SEQ ID NO: 55 - DNA, 330 bp, synthetic construct
  source 1..330: mol_type = other DNA
gacattgtgttgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcaaac

SEQ ID NO: 56 - DNA, 330 bp, synthetic construct
  source 1..330: mol_type = other DNA
gatattgtgatgacccagtctcccgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggagggggggaccaagctggaaatcaaa

SEQ ID NO: 57 - DNA, 336 bp, synthetic construct
  source 1..336: mol_type = other DNA
gacgttgtgatgacccagactccactcactttgtcggttaccattggacaaccagcctccatctcttgcaagtcaagtcagagcctcttagaaagcgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 58 - protein, 119 aa, synthetic construct
EVQLQQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSS

SEQ ID NO: 59 - protein, 115 aa, synthetic construct
EVQLQQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVSS

SEQ ID NO: 60 - protein, 113 aa, synthetic construct
DIVMTQSPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKLEIK

SEQ ID NO: 61 - protein, 110 aa, synthetic construct
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSN

SEQ ID NO: 62 - protein, 110 aa, synthetic construct
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGTKLEIK

SEQ ID NO: 63 - protein, 112 aa, synthetic construct
DVVMTQTPLTLSVTIGQPASISCKSSQSLLESDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 64 - protein, 8 aa, synthetic construct
GYIFITTW

SEQ ID NO: 65 - protein, 9 aa, synthetic construct
QNDYSYPFT

SEQ ID NO: 66 - protein, 113 aa, synthetic construct
DIVMTQSPSSLSVSAGEKVTMSCKSSQSLLNSGNQKTYLAWYQQKPGQPPKLLIYGASTRESGVPDRFTGSGSGTDFTLTISSVQAEDLAVYYCQNDYSYPFTFGSGTKLEIK

SEQ ID NO: 67 - protein, 8 aa, synthetic construct
INPSNAGT

SEQ ID NO: 68 - protein, 119 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSS

SEQ ID NO: 69 - protein, 113 aa, synthetic construct
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIK

SEQ ID NO: 70 - protein, 449 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 445 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 449 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 449 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 446 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 446 aa, synthetic construct
EVQLVQSGAEVKKPGASVKVSCKASGYTFTSYYMYWVRQAPGQGLEWIGGINPSNGGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 446 aa, synthetic construct
EVQLVQSGAEVKKPGASVKVSCKASGYTFTSYYMYWVRQAPGQGLEWIGGINPSNAGTNFNEKFKNRATLTVDKSTSTAYMELSSLRSEDTAVYYCTREGNPYYTMNYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 220 aa, synthetic construct
DIVMTQSPDSLAVSLGERATISCKSSQSLLNSGNQKTYLAWYQQKPGQPPKLLIYGASTRESGVPDRFSGSGSGTDFTLTISSLQAEDVAVYYCQNDHSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC